SEQUENCE LISTING (44 sequences)

SEQ ID NO: 1 - DNA, 28 bp, Artificial Sequence
  primer sequence
gaagcagagcattttctaatatccacaa

SEQ ID NO: 2 - DNA, 30 bp, Artificial Sequence
  primer sequence
acaagcactacaataaaggaaaatacaggg

SEQ ID NO: 3 - DNA, 25 bp, Artificial Sequence
  primer sequence
gaagcaaataggccaaaaatgaaca

SEQ ID NO: 4 - DNA, 26 bp, Artificial Sequence
  primer sequence
ttagaacagactcaaccattcctcca

SEQ ID NO: 5 - DNA, 27 bp, Artificial Sequence
  primer sequence
aagcaggggaaaataaaaacaaccaaa

SEQ ID NO: 6 - DNA, 27 bp, Artificial Sequence
  primer sequence
aagggtgttttttcttatatttctgaa

SEQ ID NO: 7 - DNA, 18 bp, Artificial Sequence
  primer sequence
agcaaaagcaggagttta

SEQ ID NO: 8 - DNA, 22 bp, Artificial Sequence
  primer sequence
aacaaggagttttttcaacgaa

SEQ ID NO: 9 - DNA, 28 bp, Artificial Sequence
  primer sequence
gcaaaagcaggggataattctattaacc

SEQ ID NO: 10 - DNA, 29 bp, Artificial Sequence
  primer sequence
agtagaaacaagggtgtttttaattaatg

SEQ ID NO: 11 - DNA, 21 bp, Artificial Sequence
  primer sequence
agcaaaagcaggagtgaagat

SEQ ID NO: 12 - DNA, 28 bp, Artificial Sequence
  primer sequence
acaaggagtttttttctaaaattgcgaa

SEQ ID NO: 13 - DNA, 27 bp, Artificial Sequence
  primer sequence
aagcaggggaaaataaaaacaaccaaa

SEQ ID NO: 14 - DNA, 27 bp, Artificial Sequence
  primer sequence
aagggtgttttttcttatatttctgaa

SEQ ID NO: 15 - DNA, 28 bp, Artificial Sequence
  primer sequence
aacaaattggaaagaaggatggaaaact

SEQ ID NO: 16 - DNA, 21 bp, Artificial Sequence
  primer sequence
ttgggccatgagctttctttg

SEQ ID NO: 17 - DNA, 24 bp, Artificial Sequence
  primer sequence
gcttatgtctctgtagtgtcttca

SEQ ID NO: 18 - DNA, 20 bp, Artificial Sequence
  primer sequence
aatgaaaccggcaatggctc

SEQ ID NO: 19 - DNA, 18 bp, Artificial Sequence
  primer sequence
agcaaaagcaggagttta

SEQ ID NO: 20 - DNA, 22 bp, Artificial Sequence
  primer sequence
aacaaggagttttttcaacgaa

SEQ ID NO: 21 - DNA, 24 bp, Artificial Sequence
  primer sequence
ctggaccagaaattccgattgtta

SEQ ID NO: 22 - DNA, 24 bp, Artificial Sequence
  primer sequence
gattggtcagggtacagcggaagt

SEQ ID NO: 23 - DNA, 24 bp, Artificial Sequence
  primer sequence
ggtcagggtacagcggaagttttg

SEQ ID NO: 24 - DNA, 28 bp, Artificial Sequence
  primer sequence
gcaaaagcaggggataattctattaacc

SEQ ID NO: 25 - DNA, 29 bp, Artificial Sequence
  primer sequence
agtagaaacaagggtgtttttaattaatg

SEQ ID NO: 26 - DNA, 23 bp, Artificial Sequence
  primer sequence
cggagcttcttgttgccctggag

SEQ ID NO: 27 - DNA, 27 bp, Artificial Sequence
  primer sequence
caaatttttcattgtttggcatagtca

SEQ ID NO: 28 - DNA, 21 bp, Artificial Sequence
  primer sequence
ctttagtagattgaattggtt

SEQ ID NO: 29 - DNA, 19 bp, Artificial Sequence
  primer sequence
ctcagaattttgatgcctg

SEQ ID NO: 30 - DNA, 21 bp, Artificial Sequence
  primer sequence
agcaaaagcaggagtgaagat

SEQ ID NO: 31 - DNA, 28 bp, Artificial Sequence
  primer sequence
acaaggagtttttttctaaaattgcgaa

SEQ ID NO: 32 - DNA, 23 bp, Artificial Sequence
  primer sequence
atgggaagaacgatcagcgagaa

SEQ ID NO: 33 - DNA, 22 bp, Artificial Sequence
  primer sequence
cccaccagcggaaagcctaatc

SEQ ID NO: 34 - DNA, 28 bp, Artificial Sequence
  primer sequence
gaagcagagcattttctaatatccacaa

SEQ ID NO: 35 - DNA, 30 bp, Artificial Sequence; identical to SEQ ID NO: 2
  primer sequence

SEQ ID NO: 36 - DNA, 28 bp, Artificial Sequence
  primer sequence
agcactcaagaggccataaacaagataa

SEQ ID NO: 37 - DNA, 23 bp, Artificial Sequence
  primer sequence
gggtcctcctggtgcattttctg

SEQ ID NO: 38 - DNA, 18 bp, Artificial Sequence
  primer sequence
ttcgcaacaatggcttgg

SEQ ID NO: 39 - DNA, 21 bp, Artificial Sequence
  primer sequence
ccagcaatagctccgaagaaa

SEQ ID NO: 40 - DNA, 25 bp, Artificial Sequence
  primer sequence
gaagcaaataggccaaaaatgaaca

SEQ ID NO: 41 - DNA, 26 bp, Artificial Sequence
  primer sequence
ttagaacagactcaaccattcctcca

SEQ ID NO: 42 - DNA, 23 bp, Artificial Sequence
  primer sequence
ccgcatgccatgatggtaaggaa

SEQ ID NO: 43 - DNA, 23 bp, Artificial Sequence
  primer sequence
ggggatggggctgtatgtcaagt

SEQ ID NO: 44 - DNA, 28 bp, Artificial Sequence
  primer sequence
ttgtttctgtctcctcttgttccattgt

(19) United States
(12) Patent Application Publication
(10) Pub. No.: US 2012/0207785 A1
(43) Pub. Date: Aug. 16, 2012

(54) PROCESS FOR PREPARING AN INFLUENZA SEED VIRUS FOR VACCINE MANUFACTURE

(76) Inventors: Luc Gilbert Fabry, Rixensart (BE); Isabelle Knott, Rixensart (BE); Ventzislav Bojidarov Vassilev, Rixensart (BE)

(21) Appl. No.: 13/503,866

(22) PCT Filed: Oct. 29, 2010
(86) PCT No.: PCT/EP10/66466

§ 371 Date: Apr. 25, 2012

(30) Foreign Application Priority Data
Oct. 30, 2009 (GB) 0919117.2

(51) Int. Cl. A61K 39/145; A61P 31/16; C12N 7/01

(52) U.S. Cl. 424/209.1; 435/235.1

(57) ABSTRACT

The present invention relates to the use of avian cell lines for the preparation of seed influenza vaccines, and to vaccines made therefrom.

### TECHNICAL FIELD

This invention is in the field of manufacturing vaccines for protecting against influenza virus.

### BACKGROUND ART

Most of the licensed inactivated influenza vaccines are produced on eggs. Field strains collected from human samples are isolated and amplified on embryonated eggs for genetic and antigenic characterization. Representative isolates are then selected and high growth seeds are prepared by reassortment or the use of reverse genetic. When seeds are released to vaccine manufactures, they have had several passages in embryonated chicken eggs.

Amplification in embryonic egg substrates may lead to selection of variants which are antigenically and biologically different from viruses isolated from the same source but amplified in mammalian cell lines i.e. MDCK cells.

Host-mediated antigenic variation has been reported, indicating selection of different subpopulations after amplification on egg or cell-based substrates (Katz; Virology 156, 386-395;(1987))

A substitution of a single amino acid in the hemagglutinin after cultivation in embryonated chicken eggs may result in a poorly immunogenic vaccine compared to an MDCK grown virus (Kodihalli & all; J of Virol, August 1995, 4888-4897)

Once egg-adapted mutants are established, growth in tissue culture does not generally result in additional changes and the original egg-adaptation mutations are maintained. Immunization of mice with Vero or MDCK grown virus compared to Egg-grown virus followed by subsequent challenge with homologous or heterologous virus suggested that the antibody response to cell-derived vaccines was more cross-reactive than the response induced by the egg-derived vaccines (Govorkova et al 1999, Dev Biol Stand. 1999;98:39-51; discussion 73-4)

The ability to replicate influenza virus without inducing significant changes in the HA protein has been demonstrated to be common to several mammalian cell lines (MDCK, MRCS, LLC-MK2 (Meyer J L Virology 1993 196:1,130-137).

It has been demonstrated that MDCK derived Influenza A/H3N2 virus induced better protection from challenge than egg derived virus in Ferrets (Katz and Webster; 1989. J. Infect. Dis.160:191-98).

A superior ability to induce cell-mediated immunity in a mouse model was observed with Vero-derived inactivated vaccine (Brühl & all; Vaccine 19; 2000 1149-1158).

Aspects of the current processes for preparing seasonal vaccines against human influenza virus and the use of cell culture techniques are described in WO2008032219. WO2008032219 discloses a number of references that support the above background position and proposes procedures using cell lines in manufacturing influenza vaccines, in which the use of eggs is preferably avoided. The most preferred cell lines are reported to be mammalian cell lines.

The present invention addresses the issues of cell culture influenza vaccine manufacture.

### STATEMENTS OF INVENTION

In one aspect the present invention relates to a process for preparing an influenza seed virus for vaccine manufacture, comprising steps of: (i) infecting a cell line with an influenza virus and (ii) amplifying the virus obtained from the infected cell line of (i) to produce influenza virus for use as a seed virus, wherein the cell line is an avian cell line.

In one aspect the present invention relates to a process for preparing an influenza seed virus for vaccine manufacture, comprising steps of: (i) infecting a cell line with an influenza virus; (ii) preparing a cDNA of at least one viral RNA segment of an influenza virus produced by the infected cell line obtained in step (i), and using the cDNA in a reverse genetics procedure to prepare a new influenza virus having at least one viral RNA segment in common with the influenza virus of step (i); and (iii) infecting a cell line with the new influenza virus, and (iv) amplifying the virus in order to produce a new influenza virus for use as a seed virus, wherein the cell line is an avian cell line for one or more of steps (i), (iii) and (iv).

In one aspect the present invention relates to a process as described above wherein the influenza virus of step (i) is obtained either directly from a patient or from a primary isolate in a cell line, or is a strain either circulating in the population or which has a hemagglutinin that is antigenically representative of an influenza virus that is circulating in the population.

In one aspect the present invention relates to a process for preparing an influenza seed virus for vaccine manufacture, comprising steps of: (i) infecting a cell line with an influenza virus (ii) passaging virus from the infected cell line obtained in step (i) at least once; and (iii) culturing the infected cell line from step (ii) in order to produce influenza virus for use as a seed virus, wherein the cell line is an avian cell line for one, or more, or all of steps (i)-(iii).

In one aspect the present invention relates to a process for preparing an influenza virus vaccine, wherein the seed virus according to any preceding claim is cultured to produce an influenza virus for vaccine manufacture, and optionally then further processed to give a vaccine.

In one aspect none of the cell lines used have a tumorigenic phenotype.

### BRIEF DESCRIPTION OF THE DRAWINGS

FIG. 1 methodology for comparing egg and cell culture processes

FIG. 2 methodology comparing egg and cell culture processes using reverse genetics

FIG. 3 comparative immunogenicity EB66-derived vs. egg derived formulations: Anti-A/Indonesia/05/2005 (H5N1) HI Titers in ferrets

FIG. 4 comparative immunogenicity EB66-derived vs. egg derived formulations: Anti-A/Indonesia/05/2005 (H5N1)NT Titers in ferrets

FIG. 5 comparative immunogenicity EB66-derived vs. egg derived formulations: Anti-A/Indonesia/05/2005 (H5N1)HI Titers in mice

FIG. 6 comparative immunogenicity EB66-derived vs. egg derived formulations: Anti-A/Indonesia/05/2005 (H5N1)NT Titers in mice

FIG. 7 comparative immunogenicity EB66-derived vs. egg derived formulations: Ferret nasal swap viral titration on MDCK: A/Solomon Island/03/2006 viral load

FIG. 8 comparative immunogenicity EB66-derived vs. egg derived and MDCK derived formulations: Ferret nasal swap viral titration on MDCK: A/Wisconsin/67/2005 viral load

FIG. 9 comparative immunogenicity EB66-derived vs. egg derived and MDCK derived formulations: Ferret nasal swap viral titration on MDCK: B/Malaysia/2506/2004 viral load

FIG. 10 comparative immunogenicity EB66-derived vs. egg derived and MDCK derived formulations: Flu type H1N1 homologous and heterologous NT Titers in ferrets

FIG. 11 comparative immunogenicity EB66-derived vs. egg derived and MDCK derived formulations: Flu type H3N2 homologous and heterologous NT Titers in ferrets

FIG. 12 comparative immunogenicity EB66-derived vs. egg derived and MDCK derived formulations: Flu type B homologous and heterologous NT Titers in ferrets

### DETAILED DESCRIPTION OF THE INVENTION

The present invention relates to the use of avian cell lines for the preparation of seed influenza vaccines, and to vaccines made thereofrom.

A seed virus, as referred to herein, is a virus which is used, directly or indirectly, to product a batch of vaccine. Generally an isolate of a virus may be used to produce a quantity of virus that comprises the master seed, which may be generally divided into aliquots and stored. An aliquot of master seed may be used to produce a volume of working seed, which is again divided into aliquots and stored. An aliquot of the working seed may then be used to produce a batch of vaccine. All batches of vaccine are then only two passages removed from the master seed.

Seed viruses that are used in the manufacture of a vaccine have suitably been characterised before use. Characterisation may include testing for contamination and/or testing for viral homogeneity. Suitably a seed virus is a single homogeneous strain.

Where referred to herein, ‘infecting’ a cell line with a virus includes transfection of that cell with viral DNA, or sufficient genetic materials to allow a functional virus to be generated within the cell.

Where referred to herein ‘amplifying’ a virus is suitably growth of a virus within a cell culture to allow the number of copies of the virus to be increased.

Where referred to herein, preparing of a cDNA of at least one viral RNA segment of an influenza virus includes the isolation, or purification or generation or otherwise obtaining of a suitable cDNA for use in a reverse genetics procedure.

Where referred to herein, a primary isolate includes to a virus isolate that has been obtained from an infected individual.

Where referred to herein, strains circulating in the population include any of those strains acknowledged by the World Health Organization (WHO) Collaborating Centers for Reference and Research on Influenza located at the Centers for Disease Control and Prevention (CDC) in Atlanta; London, United Kingdom; Melbourne, Australia; and Tokyo, Japan.

Treatment of viruses to produce a vaccine includes inactivating the influenza virus by methods well known in the art including splitting, making subunit vaccines, and chemical inactivation such as formaldehyde treatment.

The avian cell line may be directly infected with a patient sample and used to generate a primary isolate.

Alternatively the avian cell line may be infected with an influenza sample that has been previously infected into another cell line, which may be a non-human mammalian cell line such as an MDCK cell line, preferably a MDCK cell line with a non tumorigenic phenotype. In one aspect the influenza virus has not been present in eggs before use in the avian cell line.

### Avian Cell Lines

In one aspect the avian cell line is a continuous genetically stable avian cell line, such as an avian embryonic stem cell line. Suitable cell lines are chicken cell line EB14 or duck cell line EB24 or EB66 cells, such as manufactured in, or as otherwise disclosed in, WO2008129058 or WO2003076601 (produced by Vivalis: see also www.vivalis.com). A preferred cell line is EB66. These disclosures, including US equivalent publications such as US20090239297A1 and US20040058441A1, are herein fully incorporated by reference.

In one aspect continuous diploid avian cell lines are preferred, in line with the teaching of WO2008129058. In that disclosure, applicable to the present invention, the process of establishment of continuous diploid avian cell lines, named EBx®, of the invention comprises two steps: a) isolation, culture and expansion of embryonic stem cells from birds that do not contain complete endogenous proviral sequences, or a fragment thereof, susceptible to produce replication competent endogenous retroviral particles, more specifically EAV and/or ALV-E proviral sequences or a fragment thereof, in a complete culture medium containing all the factors allowing their growth and in presence of a feeder layer and supplemented with animal serum; optionally, said complete culture medium may comprise additives, such as additional amino-acids (i.e glutamine, non essential amino acids . . . ), sodium pyruvate, beta-mercaptoethanol, vitamins, protein hydrolyzate of non-animal origin (i.e yeastolate, plant hydrolyzates (soy, wheat, . . . ); b) passage by modifying the culture medium so as to obtain a total withdrawal of said factors, said feeder layer and said serum, and optionally said additives, and further obtaining adherent or suspension avian cell lines, named EBx®, that do not produce replication-competent endogenous retrovirus particles, capable of proliferating over a long period of time, in a basal medium in the absence of exogenous growth factors, feeder layer and animal serum.

More specifically, the process for obtaining continuous diploid avian cell lines derived from ES cells, wherein said avian cell lines do not produce replication competent endogenous retroviral particles, comprises the following steps of: a) isolating bird embryo(s), preferably from duck or from ev-0 chicken, at a developmental stage comprises from around stage VI of Eyal-Giladi's classification (EYAL-GILADI's classification: EYAL-GILADI and KOCHAN, 1976, <<From cleavage to primitive streack formation: a complementary normal table and a new look at the first stages of the development in the chick>>. “General Morphology” Dev. Biol., 49:321-337) and before hatching, preferably around oviposition, wherein the genome of said bird does not contain endogenous proviral sequences susceptible to produce replication competent endogenous retroviral particles; b) suspending avian embryonic stem (ES) cells obtained by dissociating embryo(s) of step a) in a basal culture medium supplemented with: —Insulin Growth factor 1 (IGF-1) and Ciliary Neurotrophic factor (CNTF); —animal serum; and —optionally, growth factors selected in the group comprising interleukin 6 (IL-6), interleukin 6 receptor (IL-6R), Stem cell Factor (SCF) and Fibroblast Growth Factor (FGF); c) seeding the suspension of ES cells obtained in step b) on a layer of feeder cells and further culturing the ES cells for at least one passage; d) optionally withdrawing all the growth factors selected from the group comprising IL-6, IL-6R, SCF, FGF from the culture medium over a range of several passages from 1 to around 15 passages, preferably from 3 to around 15 passages and further culturing the avian ES cells for at least one passage. Preferably, the withdrawing of all the growth factors selected from the group comprising IL-6, IL-6R, SCF, FGF from the culture medium is performed simultaneously over one passage. Usually, the withdrawing of IL-6, IL-6R, SCF, FGF is performed at around passage 10 to 15; e) withdrawing IGF-1 and CNTF from the culture medium and further culturing the avian ES cells for at least one passage. Preferably, the withdrawing of the growth factors selected from the group comprising IGF-1 and CNTF from the culture medium is performed simultaneously, over one passage. Usually, the withdrawing of IGF-1 and CNTF is performed at around passage NO 15 to NO 25. Alternatively, the withdrawing of IGF-1 and CNTF is performed by progressive decreasing over several passages (at least 2 passages and approximately up to 15 passages); f) progressively decreasing the concentration of feeder cells in the culture medium so as to obtain a total withdrawal of feeder layer after several passages, and further culturing the cells; g) optionally, progressively decreasing the concentration of additives in the culture medium so as to obtain a total withdrawal of additives after at least one passage; and, h) optionally, progressively decreasing the concentration of animal serum in the culture medium so as to obtain a total withdrawal of animal serum after several passages; and, i) obtaining adherent avian cell lines, named EBx®, derived from ES cells capable of proliferating in a basal medium in the absence of growth factors, feeder layer optionally without animal serum and additives, and wherein said continuous diploid avian cell lines do not produce replication-competent endogenous retrovirus particles; j) optionally, further adapting said adherent avian EBx® cell lines to suspension culture conditions. The step of adaptation of cell culture to suspension can take place all along the process of establishment of EBx® cells. For example, with duck EBx® cells derived from Muscovy embryonic stem cells, the cells were adapted to the growth in suspension prior feeder layer withdrawal. For duck EB® cells (EB24, EB26, EB66) derived from Pekin duck, the cells were adapted to the growth in suspension prior animal serum withdrawal.

k) Optionally further subcloning said avian EBx® cells, for example by limit dilution.

In another aspect the process for obtaining continuous diploid avian cell lines, named EBx®, derived from avian embryonic stem cells (ES), wherein said avian cell lines do not produce replication-competent endogenous retrovirus particles, comprises the steps of: a) isolating bird embryo(s) at a developmental stage around oviposition, wherein the genome of said bird does not contain endogenous proviral sequences susceptible to produce replication competent endogenous retroviral particles; b) suspending avian embryonic stem (ES) cells obtained by dissociating embryo(s) of step a) in a basal culture medium supplemented with at least: —Insulin Growth factor 1 (IGF-1) and Ciliary Neurotrophic factor (CNTF); and —mammalian serum such as foetal bovine serum; c) seeding the suspension of ES cells obtained in step b) on a layer of feeder cells and further culturing the ES cells for at least one passage; e) withdrawing IGF-1 and CNTF from the culture medium, and further culturing the cells for at least one passage; f) progressively decreasing the concentration of feeder cells in the culture medium so as to obtain a total withdrawal of feeder layer after several passages, and further culturing the cells; g) progressively decreasing the concentration of said mammalian serum in the culture medium so as to obtain a total withdrawal of mammalian serum after several passages and: h) obtaining adherent avian EBx® cell lines derived from ES cells capable of proliferating in a basal medium in the absence of growth factors, feeder layer and mammalian serum, and wherein said continuous diploid avian cell lines do not produce replication-competent endogenous retrovirus particles; i) optionally, further adapting adherent avian EBx® cell lines to suspension culture conditions, preferably by promoting the growth as suspension, more preferably by transferring the adherent avian EBx® cell lines obtained in step h) in another support having lower attachment characteristic than the initial support (i.e. such as Ultra Low attachment support).

Step j) of adapting adherent avian EBx® cell lines to suspension culture conditions, when carried out, can be effected in another preferred embodiment before the step g) of progressively decreasing the concentration of mammalian serum in the culture medium.

In another preferred embodiment, the basal culture medium in step b) of the process for obtaining continuous diploid avian cell lines according to the present invention, is further supplemented with a growth factor selected in the group comprising interleukin 6 (IL-6), interleukin 6 receptor (IL-6R), Stem cell Factor (SCF) and Fibroblast Growth Factor (FGF), and the said process further comprises a step d) of: d) optionally withdrawing all the growth factors selected from the group comprising IL-6, IL-6R, SCF, FGF from the culture medium and further culturing the ES cells for at least one passage.

In a more preferred embodiment, when step d) is carried out, the step e) of withdrawing IGF-1 and CNTF from the culture medium, is effected after the step d) of withdrawing all the growth factors selected from the group comprising IL-6, IL-6R, SCF, FGF from the culture medium.

In one aspect the avian cell are able to grow in suspension. In one aspect the avian cells are able to grow in serum-free media.

In one aspect the avian cell have no in vivo tumourogenicity.

In one aspect the avian cell line shares with mammalian cell lines α2-6 sialic acid receptors.

Suitable media for the growth of EB66 is EX-CELL EBx media from SAFC biosciences.

Such avian cell lines may be used for the direct isolation of influenza viruses, or may be infected with suitable influenza strains for vaccine manufacture, such as strains either circulating in the population or which have a hemagglutinin that is antigenically representative of an influenza virus that is circulating in the population.

In one aspect of the invention only avian cell lines are used in the preparation of seed vaccine from a patient sample, and no other types of cell line, although the use of different avian cell lines within this process is specifically contemplated. In one aspect of the invention only avian cell lines are used in the preparation of the vaccine, and no other types of cell line.

Thus in one aspect the influenza virus is infected directly into avian cells, suitably EB66 cells, from a patient sample (e.g. a clinical isolate) and used to make a vaccine seed without the use of eggs or mammalian cell culture.

In one aspect the influenza virus is infected directly into avian cells, suitably EB66 cells, from a patient sample (e.g. a clinical isolate) and then infected again into avian cells, optionally of a different type, to make a vaccine seed.

In one aspect the influenza virus is passaged through an avian cell line once or twice, and in one aspect not more than two passage steps are carried out, before the virus is grown up for antigen harvesting and vaccine preparation.

In one aspect, when the influenza strain is an H3N2 strain, in particular where the strain the A/Wisconsin/67/2005(H3N2)strain, then the influenza virus is not passaged more than twice.

### Use of Multiple Different Cell Lines

The avian cell line including the specific cell lines mentioned above may be infected with an influenza sample that has been already previously been infected into a cell line, which cell line may comprise mammalian cells that support viral replication, for example, hamster, cattle, primate (including humans and monkeys) and dog cells, although the use of human and other primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line. Suitable dog cells are e.g. kidney cells, as in the CLDK and MDCK cell lines. Suitable cell lines include, but are not limited to: MDCK; CHO; CLDK; HKCC; 293T; BHK; Vero; MRC-5; PER.C6; FRhL2; WI-38; etc. Suitable cell lines are widely available e.g. from the American Type Cell Culture (ATCC) collection, and the European Collection of Cell Cultures (ECACC). Any of these cell types can be used for growth, reassortment and/or passaging according to the invention. MDCK cell lines present different tumorigenic phenotypes. ATCC cell line CCL-34 is described by Krause as non tumorigenic. Other MDCK cells, in particular, cells adapted to suspension display a tumorigenic phenotype. Some clones of MDCK were found non tumorigenic such as BV5F1 (WO05/113758).

In one aspect the influenza virus is infected directly into non-human mammalian cells, suitably MDCK cells, from a patient sample and then infected into avian cells, suitably avian embryonic stem cells such as chicken cells EB14 or duck cells EB24 or EB66 cells, to make a virus seed for use in a vaccine production.

In one aspect the influenza virus is infected directly into avian cells, suitably EB66 cells, from a patient sample and then infected into non-human mammalian cells, suitably MDCK cells, and preferably MDCK cells having a non tumorigenic phenotype, to make a virus seed for use in a vaccine production.

### General (Non-Avian Cell Specific Features)

Suitable background information describing influenza virus strains, mammalian cell lines, reverse genetics techniques, vaccine preparation methodologies, influenza receptor binding, and pharmaceutical compositions is provided in WO2008032219, the teaching of which is incorporated herein by reference.

In one aspect of the invention the seed virus may be sequenced; and/or used to elicit antisera; and/or used to prepare working seed lots.

In one aspect the seed virus genome has no PR/8/34 segments.

In one aspect the seed virus has a hemagglutinin with a binding preference for oligosaccharides with a Sia([alpha]2,6)Gal terminal disaccharide compared to oligosaccharides with a Sia([alpha]2,3)Gal terminal disaccharide.

In one aspect the process of the invention involves (i) infecting a cell line with an influenza virus and (ii) passaging virus from the infected cell line obtained in step (i) at least once; before amplifying the virus. A passaging step will typically involve: allowing the influenza virus to replicate in cell culture; collecting replicated virus e.g. from the culture supernatant; and transferring the collected replicated virus to an uninfected cell culture. This process can be repeated. After at least one passage, the virus is allowed to replicate and virus is collected for use as a seed virus.

### Vaccine

In one aspect the invention also relates to a process for preparing an influenza virus vaccine, wherein the seed virus made as disclosed herein is cultured to produce influenza virus for vaccine manufacture. The virus may then be treated to produce a vaccine, for example by producing an inactivated whole vaccine, split vaccine, subunit vaccine including sub-unit vaccines based on recombinantly expressed influenza virus antigens such as HA, VLP or virosomal based vaccine. Methods for producing such vaccines are well known in the art, and described further below.

In one aspect a vaccine so produced contains less than 25 ng, suitably less than 10 ng of residual host cell DNA per dose.

In one aspect a multi-valent influenza vaccine may be produced by manufacturing a vaccine as disclosed herein for a single strain and then mixing that vaccine with one or more other individual vaccines, optionally also produced using the method of the invention to make the multi-valent influenza vaccine. In one aspect the multi-valent influenza vaccine may have two influenza A virus strains and one influenza B virus strain.

In one aspect the vaccine comprises at least one pandemic strain.

In one aspect the vaccine is substantially free from mercury.

In one aspect the vaccine includes an adjuvant.

### Adjuvant

In one aspect a pharmaceutical composition of the invention is not adjuvanted. In another aspect a pharmaceutical composition of the invention comprises an adjuvant.

### Oil-In-Water Emulsion Adjuvant

In one aspect an adjuvant according to the present invention is an emulsion, in particular, an oil-in-water emulsion, and may optionally comprise other immunostimulants. In particular, the oil phase of the emulsion system comprises a metabolisable oil. The meaning of the term metabolisable oil is well known in the art. Metabolisable can be defined as “being capable of being transformed by metabolism” (Dorland's Illustrated Medical Dictionary, W. B. Sanders Company, 25^{th} edition (1974)). The oil may be any vegetable oil, fish, oil, animal or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others. A particularly suitable metabolisable oil is squalene. Squalene (2,6,10,15,19, 23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is an oil for use in this invention. Squalene is a metabolisable oil by virtue of the fact that it is an intermediate in the biosynthesis of cholesterol (Merck index, 10^{th} Edition, entry no.8619).

Oil-in-water emulsions per se are well known in the art, and have been suggested to be useful as adjuvant compositions (EP399843B); also combinations of oil-in-water emulsions and other active agents have been described as adjuvants for vaccines: WO95/17210; WO98/56414; WO99/12565; WO99/11241; WO2006/100109; WO 2006/100110; WO 2006/100111; WO2008/128939; WO2008/043774 which disclose emulsion adjuvants based on squalene, a-tocopherol, and TWEEN 80, optionally formulated with the immunostimulants QS21 and/or 3D-MPL). Other oil-in-water emulsion-based adjuvants have been described, such as those disclosed in WO90/14837; WO00/50006; WO2007/052155; WO2007/080308; WO2007/006939, all of which form oil emulsion systems (in particular when based on squalene) to form alternative adjuvants and compositions of the present invention.

In a specific embodiment, an oil-in-water emulsion comprises a metabolisable, non-toxic oil, such as squalane or squalene, optionally a tocol such as tocopherol in particular alpha tocopherol (and optionally both squalene and alpha tocopherol) and an emulsifier (or surfactant) such as the non-ionic surfactant TWEEN 80™ or Polysorbate 80. In a specific embodiment the oil emulsion further comprises a sterol such as cholesterol. Mixtures of surfactants can be used e.g. Tween 80 (or Polysorbate 80™)/Span 85 mixtures, or Tween80 (or Polysorbate 80™)/Triton-X1 00 mixtures.

Tocols (e.g. vitamin E) are also used in oil emulsions adjuvants (EP0382271B1; U.S. Pat. No. 5,667,784; WO95/17210). Tocols used in oil emulsions (optionally oil-in-water emulsions) may be formulated as described in EP0382271B1, in that the tocols may be dispersions of tocol droplets, optionally comprising an emulsifier, of optionally less than 1 micron in diameter. Alternatively, the tocols may be used in combination with another oil, to form the oil phase of an oil emulsion. Examples of oil emulsions which may be used in combination with the tocol are described herein, such as the metabolisable oils described above.

The method of producing oil-in-water emulsions is well known to the person skilled in the art. Commonly, the method comprises mixing the oil phase with a surfactant such as a PBS/TWEEN80™ solution, followed by homogenisation using a homogenizer. It would be clear to a man skilled in the art that a method comprising passing the mixture twice through a syringe needle would be suitable for homogenising small volumes of liquid. Equally, the emulsification process in microfluidiser (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier should be in an aqueous carrier. The aqueous carrier may be, for example, phosphate buffered saline.

The size of the oil droplets found within a stable oil-in-water emulsion are optionally less than 1 micron, may be in the range of substantially 30-600 nm, optionally substantially around 30-500 nm in diameter, and optionally substantially 150-500 nm in diameter, and in particular about 150 nm in diameter as measured by photon correlation spectroscopy. In this regard, 80% of the oil droplets by number should be within the ranges, optionally more than 90% and optionally more than 95% of the oil droplets by number are within the defined size ranges.

In one aspect, the adjuvant of the immunogenic compositions are comprises a submicron oil-in-water emulsion of the following composition:

from 2 to 10% squalene, from 0.3 to 3% TWEEN80™ and optionally, from 2 to 10% alpha-tocopherol;
about 5% squalene, about 0.5% polysorbate 80 and about 0.5% Span 85. This adjuvant is called MF59.

In another aspect components present in the adjuvant of the immunogenic compositions are at lower amounts than had previously been thought useful, suitably at below 11 mg metabolisable oil (such as squalene), for example between 0.5-11 mg, 0.5-10 mg or 0.5-9 mg, and at below 5 mg emulsifying agent (suitably such as polyoxyethylene sorbitan monooleate), for example between 0.1-5 mg, per human dose of the immunogenic composition. Suitably tocol (e.g. alpha-tocopherol) where present is at below 12 mg, for example between 0.5-12 mg. An adjuvant composition of the invention comprises an oil-in-water emulsion adjuvant, suitably comprising a metabolisable oil in an amount of between 0.5-10 mg, and an emulsifying agent in an amount of between 0.4-4 mg, and optionally a tocol in an amount of between 0.5-11 mg. Another adjuvant composition of the invention comprises an oil-in-water emulsion adjuvant, suitably comprising a metabolisable oil in an amount of between 1-7 mg, and an emulsifying agent in an amount of between 0.3-3 mg, and optionally a tocol in an amount of between 1-8 mg. Suitably said emulsion has oil droplets of which at least 70%, suitably at least 80% by intensity have diameters of less than 1 μm.

By the term “human dose” is meant a influenza composition dose (after mixing the adjuvant and the antigen components) which is delivered in a volume suitable for human use. Generally this is between 0.25 and 1.5 ml. In one embodiment, a human dose is about 0.5 ml. In a further embodiment, a human dose is higher than 0.5 ml, for example about 0.6, 0.7, 0.8, 0.9 or about 1 ml. In a further embodiment, a human dose is between 1 ml and 1.5 ml. In another embodiment, in particular when the immunogenic composition is for the paediatric population, a human dose may be less than 0.5 ml, for example between 0.25 and 0.5 ml or exactly 0.1 ml, 0.2 ml, 0.25 ml, 0.3 ml or 0.4 ml. The invention is characterised in that each or all of the individual components of the adjuvant within the immunogenic composition is/are at a lower level than previously thought useful and is/are typically as recited above. Particularly suitable compositions comprise the following o/w adjuvant components in the following amounts in the final volume of human dose (suitably of about 0.5 ml or about 0.7 ml) (Table 1 and Table 2):

TABLE 1
specific o/w emulsion-based adjuvants suitable for use in the invention

| | Adjuvant | Adjuvant | Adjuvant | Adjuvant | Adjuvant | Adjuvant | Adjuvant | Adjuvant |
|---|---|---|---|---|---|---|---|---|
| | A | B | E | F | C | H | G | D |
| o/w emulsion* | 125 μl | 100 μl | 83.33 μl | 62.5 μl | 50 μl | 41.67 μl | 31.25 μl | 25 μl |
| Components: | | | | | | | | |
| Tocopherol (mg) | 5.94 | 4.75 | 3.96 | 2.97 | 2.38 | 1.98 | 1.49 | 1.19 |
| | (5.6-6.2) | (4.5-5.0) | (3.8-4.2) | (2.8-3.1) | (2.3-2.5) | (1.9-2.1) | (1.4-1.6) | (1.1-1.25) |
| Squalene (mg) | 5.35 | 4.28 | 3.57 | 2.68 | 2.14 | 1.78 | 1.34 | 1.07 |
| | (5.1-5.6) | (4.1-4.5) | (3.4-3.75) | (2.5-2.8) | (2.0-2.25) | (1.7-1.9) | (1.3-1.4) | (1.0-1.1) |
| Polysorbate 80 or | 2.43 | 1.94 | 1.62 | 1.21 | 0.97 | 0.81 | 0.61 | 0.49 |
| Tween 80 (mg) | (2.3-2.6) | (1.8-2.0) | (1.5-1.7) | (1.1-1.3) | (0.9-1.0) | (0.8-0.9) | (0.58-0.64) | (0.47-0.51) |

*suitable o/w volumes are given for illustration but are not limiting
( ) between brackets are suitable ranges (+/−5%) around the above value

TABLE 2
specific o/w emulsion-based adjuvants suitable for use in the invention

| | Adjuvant | Adjuvant | Adjuvant | Adjuvant | Adjuvant | Adjuvant | Adjuvant | Adjuvant |
|---|---|---|---|---|---|---|---|---|
| | I | J | K | L | M | N | O | P |
| Components: | | | | | | | | |
| Span 85 (mg) | 0.59 | 0.47 | 0.39 | 0.29 | 0.24 | 0.2 | 0.15 | 0.12 |
| | (0.56-0.62) | (0.45-0.49) | (0.37-0.41) | (0.28-0.3) | (0.23-0.25) | (0.19-0.21) | (0.14-0.16) | (0.11-0.13) |
| Squalene (mg) | 4.88 | 3.9 | 3.25 | 2.44 | 1.95 | 1.63 | 1.22 | 0.98 |
| | (4.6-5.1) | (3.7-4.1) | (3.1-3.4) | (2.3-2.6) | (1.85-2.05) | (1.55-1.7) | (1.2-1.3) | (0.9-1.1) |
| Polysorbate 80 or | 0.59 | 0.47 | 0.39 | 0.29 | 0.24 | 0.2 | 0.15 | 0.12 |
| Tween 80 (mg) | (0.56-0.62) | (0.45-0.49) | (0.37-0.41) | (0.28-0.3) | (0.23-0.25) | (0.19-0.21) | (0.14-0.16) | (0.11-0.13) |
| | Adjuvant | Adjuvant | Adjuvant | Adjuvant | Adjuvant | Adjuvant | Adjuvant | Adjuvant |
| | Q | R | S | T | U | V | W | X |
| Components: | | | | | | | | |
| Span 85 (mg) | 0.84 | 0.67 | 0.56 | 0.42 | 0.34 | 0.28 | 0.21 | 0.17 |
| | (0.8-0.9) | (0.64-0.7) | (0.53-0.6) | (0.4-0.44) | (0.32-0.36) | (0.27-0.3) | (0.2-0.22) | (0.16-0.18) |
| Squalene (mg) | 4.88 | 3.9 | 3.25 | 2.44 | 1.95 | 1.63 | 1.22 | 0.98 |
| | (4.6-5.1) | (3.7-4.1) | (3.1-3.4) | (2.3-2.6) | (1.85-2.05) | (1.55-1.7) | (1.2-1.3) | (0.9-1.1) |
| Polysorbate 80 or | 0.84 | 0.67 | 0.56 | 0.42 | 0.34 | 0.28 | 0.21 | 0.17 |
| Tween 80 (mg) | (0.8-0.9) | (0.64-0.7) | (0.53-0.6) | (0.4-0.44) | (0.32-0.36) | (0.27-0.3) | (0.2-0.22) | (0.16-0.18) |

( ) between brackets are suitable ranges (+/−5%) around the above value (+/−5%)

All numerical values given (e.g. in % or in mg) including those in Tables 1 and 2 should be understood to allow for a 5% variation, i.e. 4.88 mg squalene should be understood to mean between 4.64-5.12 mg.

A pre-dilution of each component (o/w emulsion and antigen) may be performed to produce an adjuvanted vaccine that delivers the required HA amount and the required adjuvant component amount.

In one embodiment, the liquid adjuvant is used to reconstitute a lyophilised influenza antigen composition. In this embodiment, the human dose suitable volume of the adjuvant composition is approximately equal to the final volume of the human dose. The liquid adjuvant composition is added to the vial containing the lyophilised antigen composition. The final human dose can vary between 0.5 and 1.5 ml.

Optionally the ratio of oil (e.g. squalene): tocol (e.g. a-tocopherol) is equal or less than 1 as this provides a more stable emulsion.

In some cases it may be advantageous that the vaccines of the present invention will further contain a stabilizer, suitably a derivative of alpha-tocopherol such as alpha-tocopherol succinate.

The dose of the components at which the adjuvant is operated in the immunogenic composition is suitably able to enhance an immune response to an antigen in a human. In particular a suitable amount of metabolisable oil, tocol and polyoxyethylene sorbitan monooleate is that which improves the immunological potential of the composition compared to the unadjuvanted composition, or is that which gives an immunological potential similar to that obtained with the composition adjuvanted with an adjuvant comprising another—higher—amount of said components, in a target human population, whilst being acceptable from a reactogenicity profile.

### Optional Immunostimulants

In one aspect the adjuvant is an oil-in-water emulsion adjuvant comprising a metabolisable oil such as squalene, a tocol such as alpha-tocopherol and a surfactant such as polysorbate 80, in the amounts defined above, and does not contain any additional immunostimulants(s), in particular it does not contain a non-toxic lipid A derivative (such as 3D-MPL) or a saponin (such as QS21).

In another aspect of the invention there is provided a vaccine composition comprising an antigen or antigen composition and an adjuvant composition comprising an oil-in-water emulsion and optionally one or more further immunostimulants. Additional immunostimulatants may non-toxic lipid A derivatives (such as 3D-MPL) or a saponin (such as QS21). In another aspect of the invention an oil-in-water emulsion adjuvant optionally comprises one or more additional adjuvants or immunostimulants other than QS21 and/or MPL.

In another aspect of the invention the pharmaceutical composition of the invention comprises adjuvants other than oil in water adjuvants, including any component listed herein, either alone or in combination for example non-toxic lipid A derivative (such as 3D-MPL) or a saponin (such as QS21).

In another aspect of the invention, the adjuvant comprises a lipopolysaccharide, suitably a non-toxic derivative of lipid A, particularly monophosphoryl lipid A or more particularly 3-Deacylated monophoshoryl lipid A (3D-MPL). 3D-MPL is sold under the name MPL by GlaxoSmithKline Biologicals N.A. and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-g (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL may be used. Small particle 3D-MPL has a particle size such that it may be sterile-filtered through a 0.22 μm filter. Such preparations are described in WO94/21292.

Said lipopolysaccharide, such as 3D-MPL, can be used at amounts between 1 and 50 μg, per human dose of the immunogenic composition. Such 3D-MPL can be used at a level of about 25 μg. In another embodiment, the human dose of the immunogenic composition comprises 3D-MPL at a level of about 10 μg, for example between 5 and 15 μg. In a further embodiment, the human dose of the immunogenic composition comprises 3D-MPL at a level of about 5 μg.

In another embodiment, synthetic derivatives of lipid A are used as optional additional immunostimulant, some being described as TLR-4 agonists, and include, but are not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM 294 DP (3S, 9 R)-3-[(R)-dodecanoyloxytetradecanoylamino]-4-oxo-5-aza-9(R)-[(R)-3-hydroxytetradecanoylamino]decan-1,10-diol, 1,10-bis(dihydrogenophosphate) (WO99/64301 and WO 00/0462)
OM 197 MP-Ac DP (3S-, 9R) -3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signalling response through TLR-4 (Sabroe et al, JI 2003 p1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, muramyl dipeptide (MDP) or F protein of respiratory syncitial virus. In one embodiment the TLR agonist is HSP 60, 70 or 90. Other suitable TLR-4 ligands are as described in WO 2003/011223 and in WO 2003/099195 such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. Suitably said TLR-4 ligand is ER804057.

Toll-like receptors (TLRs) are type I transmembrane receptors, evolutionarily conserved between insects and humans. Ten TLRs have so far been established (TLRs 1-10) (Sabroe et al, JI 2003 p1630-5). Members of the TLR family have similar extracellular and intracellular domains; their extracellular domains have been shown to have leucine—rich repeating sequences, and their intracellular domains are similar to the intracellular region of the interleukin—1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NF-KB activation of cytokines; this Myd88 pathway is one way by which cytokine release is effected by TLR activation. The main expression of TLRs is in cell types such as antigen presenting cells (e.g. dendritic cells, macrophages etc).

Activation of dendritic cells by stimulation through the TLRs leads to maturation of dendritic cells, and production of inflammatory cytokines such as IL-12. Research carried out so far has found that TLRs recognise different types of agonists, although some agonists are common to several TLRs. TLR agonists are predominantly derived from bacteria or viruses, and include molecules such as flagellin or bacterial lipopolysaccharide (LPS). By “TLR agonist” it is meant a component which is capable of causing a signalling response through a TLR signalling pathway, either as a direct ligand or indirectly through generation of endogenous or exogenous ligand (Sabroe et al, JI 2003 p1630-5).

In another embodiment, other natural or synthetic agonists of TLR molecules are used as optional additional immunostimulants. These could include, but are not limited to agonists for TLR2, TLR3, TLR7, TLR8 and TLR9.

Accordingly, in one embodiment, the adjuvant composition comprises an immunostimulant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In one embodiment of the present invention, a TLR agonist is used that is capable of causing a signalling response through TLR-1 (Sabroe et al, JI 2003 p1630-5). Suitably, the TLR agonist capable of causing a signalling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam₃Cys) LP which mimics the acetylated amino terminus of a bacterial lipoprotein and OspA LP from Borrelia burgdorfei.

In an alternative embodiment, a TLR agonist is used that is capable of causing a signalling response through TLR-2 (Sabroe et al, JI 2003 p1630-5). Suitably, the TLR agonist capable of causing a signalling response through TLR-2 is one or more of a lipoprotein, a peptidoglycan, a bacterial lipopeptide from M tuberculosis, B burgdorferi. T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, a TLR agonist is used that is capable of causing a signalling response through TLR-3 (Sabroe et al, JI 2003 p1630-5). Suitably, the TLR agonist capable of causing a signalling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signalling response through TLR-5 (Sabroe et al, JI 2003 p1630-5). Suitably, the TLR agonist capable of causing a signalling response through TLR-5 is bacterial flagellin.

In an alternative embodiment, a TLR agonist is used that is capable of causing a signalling response through TLR-6 (Sabroe et al, JI 2003 p1630-5). Suitably, the TLR agonist capable of causing a signalling response through TLR-6 is mycobacterial lipoprotein, di-acylated LP, and phenol-soluble modulin. Further TLR6 agonists are described in WO2003043572.

In an alternative embodiment, a TLR agonist is used that is capable of causing a signalling response through TLR-7 (Sabroe et al, JI 2003 p1630-5). Suitably, the TLR agonist capable of causing a signalling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO02085905.

In an alternative embodiment, a TLR agonist is used that is capable of causing a signalling response through TLR-8 (Sabroe et al, JI 2003 p1630-5). Suitably, the TLR agonist capable of causing a signalling response through TLR-8 is a single stranded RNA (ssRNA), an imidazoquinoline molecule with anti-viral activity, for example resiquimod (R848); resiquimod is also capable of recognition by TLR-7. Other TLR-8 agonists which may be used include those described in WO2004071459.

In an alternative embodiment, a TLR agonist is used that is capable of causing a signalling response through TLR-9 (Sabroe et al, JI 2003 p1630-5). In one embodiment, the TLR agonist capable of causing a signalling response through TLR-9 is bacterial or viral DNA, DNA containing unmethylated CpG nucleotides, in particular sequence contexts known as CpG motifs (WO 96/02555, WO 99/33488, U.S. Pat. No. 6,008,200 and U.S. Pat. No. 5,856,462). Suitably, CpG nucleotides are CpG oligonucleotides. The CpG oligonucleotides of the present invention are typically deoxynucleotides. In a specific embodiment the internucleotide in the oligonucleotide is phosphorodithioate, or suitably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204. A preferred oligonucleotide is CpG7909 disclosed in WO2008/128939.

In another embodiment, the adjuvant composition comprises a saponin adjuvant. A particularly suitable saponin for use in the present invention is Quil A and its derivatives such as QS21 (EP 0 362 278), also known as QA21. QS-21 is a natural saponin derived from the bark of Quillaja saponaria Molina, which induces CD8+ cytotoxic T cells (CTLs), Th1 cells and a predominant IgG2a antibody response and is a preferred saponin in the context of the present invention. Said immunologically active saponin, such as QS21, can be used in amounts of between 1 and 50 μg, per human dose of the immunogenic composition.

The dose of 3D-MPL and/or QS21 is suitably able to enhance an immune response to an antigen in a human. In particular a suitable 3D-MPL and/or QS21 amount is that which improves the immunological potential of the composition compared to the unadjuvanted composition, or compared to the composition adjuvanted with another 3D-MPL or QS21 amount, whilst being acceptable from a reactogenicity profile. Typically for human administration the saponin (e.g. QS21) and/or LPS derivative (e.g. 3D-MPL) will be present in a human dose of immunogenic composition in the range of 1 μg-200 μg, such as 10-50 μg, or 1 μg-25 μg per dose.

In a specific embodiment, the adjuvant and immunogenic compositions according to the invention comprise a saponin (e.g. QS21) and/or an LPS derivative (e.g. 3D-MPL) in an oil emulsion described above, together with a sterol (e.g. cholesterol). These sterols are well known in the art, for example cholesterol is disclosed in the Merck Index, 11th Edn., page 341, as a naturally occurring sterol found in animal fat. Additionally the oil emulsion (in particular the oil-in-water emulsion) may contain Span 85 and/or lecithin and/or tricaprylin. Adjuvants comprising an oil-in-water emulsion, a sterol and a saponin are described in WO 99/12565. Examples of further immunostimulants are described herein and in “Vaccine Design—The Subunit and Adjuvant Approach” 1995, Pharmaceutical Biotechnology, Volume 6, Eds. Powell, M. F., and Newman, M. J., Plenum Press, New York and London, ISBN 0-306-44867-X.

Where squalene and a saponin (optionally QS21) are included, it is of benefit to also include a sterol (optionally cholesterol) to the formulation as this allows a reduction in the total level of oil in the emulsion. This leads to a reduced cost of manufacture, improvement of the overall comfort of the vaccination, and also qualitative and quantitative improvements of the resultant immune responses, such as improved IFN-γ production. Accordingly, the adjuvant system of the present invention typically comprises a ratio of metabolisable oil:saponin (w/w) in the range of 200:1 to 300:1, also the present invention can be used in a “low oil” form the optional range of which is 1:1 to 200:1, optionally 20:1 to 100:1, or substantially 48:1, this vaccine retains the beneficial adjuvant properties of all of the components, with a much reduced reactogenicity profile. Accordingly, some embodiments have a ratio of squalene:QS21 (w/w) in the range of 1:1 to 250:1, or 20:1 to 200:1, or 20:1 to 100:1, or substantially 48:1. Optionally a sterol (e.g. cholesterol) is also included present at a ratio of saponin:sterol as described herein.

Adjuvants wherein an additional immunostimulant is optionally included are particularly suitable for infant and/or elderly vaccine formulations.

In another aspect an oil-in-water emulsion adjuvant may optionally further comprise 5-60, 10-50, or 20-30 μg (e.g. 5-15, 40-50, 10, 20, 30, 40 or 50 μg) lipid A derivative (for instance 3D-MPL).

### Influenza Vaccine Manufacture Processes

In one embodiment, the vaccine of the present invention may be an inactivated split vaccine, whole vaccine, subunit vaccine including sub-unit vaccines based on recombinantly expressed influenza virus antigens such as HA or virosome or VLP based vaccine.

A split influenza virus vaccine for use according to the present invention suitably comprises a split virus or split virus antigenic preparation, where virus particles are disrupted with detergents or other reagents to solubilise the lipid envelope. Split virus or split virus antigenic preparations thereof are suitably prepared by fragmentation of whole influenza virus, either infectious or inactivated, with solubilising concentrations of organic solvents or detergents and subsequent removal of all or the majority of the solubilising agent and some or most of the viral lipid material. By split virus antigenic preparation thereof is meant a split virus preparation which may have undergone some degree of purification compared to the split virus whilst retaining most of the antigenic properties of the split virus components. For example, when produced in cell culture, the split virus may be depleted from host cell contaminants. A split virus antigenic preparation may comprise split virus antigenic components of more than one viral strain. Vaccines containing split virus (called ‘influenza split vaccine’) or split virus antigenic preparations generally contain residual matrix protein and nucleoprotein and sometimes lipid, as well as the membrane envelope proteins. Such split virus vaccines will usually contain most or all of the virus structural proteins although not necessarily in the same proportions as they occur in the whole virus. Examples of commercially available split vaccines are for example FLUARIX™, FLUSHIELD™, or FLUZONE™.

In another embodiment, the influenza virus vaccine is in the form of a purified sub-unit influenza vaccine. Sub-unit influenza vaccines generally contain the two major envelope proteins, HA and NA, and may have an additional advantage over whole virion vaccines as they are generally less reactogenic, particularly in young vaccinees. Sub-unit vaccines can be purified from disrupted viral particles. Examples of commercially available sub-unit vaccines are for example AGRIPPAL™, or FLUVIRIN™. In a specific embodiment, sub-unit vaccines are prepared from at least one major envelope component such as from haemagglutinin (HA), neuraminidase (NA), or M2, suitably from HA. Suitably they comprise combinations of two antigens or more, such as a combination of at least two of the influenza structural proteins HA, NA, Matrix 1 (M1) and M2, suitably a combination of both HA and NA, optionally comprising M1.

Alternatively, the influenza virus vaccine may be in the form of a whole virus vaccine.

In one embodiment, the influenza virus vaccine comprises virosomes. Virosomes are spherical, unilamellar vesicles which retain the functional viral envelope glycoproteins HA and NA in authentic conformation, intercalated in the virosomes' phospholipids bilayer membrane. Examples of commercially available virosomal vaccines are for example INFLEXAL V™, or INVAVAC™.

In another embodiment, the sub-unit influenza components are expressed in the form of virus-like-particles (VLP) or capsomers, suitably plant-made or insect cells-made VLPs. VLPs present the antigens in their native form. The VLP sub-unit technology may be based entirely on influenza proteins, or may rely on other virus such as the murine leukaemia virus (MLV) and may therefore comprise a non-influenza antigen such as MLV gag protein. A suitable VLP comprises at least one, suitably at least two influenza proteins, optionally with other influenza or non-influenza proteins, such as M1 and HA, HA and NA, HA, NA and M1 or HA, NA and MLV gag. It may be produced either in plant cells or insect cells. VLPs can also carry antigens from more than one influenza strain, such as VLPs made from two seasonal strains (e.g. H1 N1 and H3N2) or from one seasonal and one pandemic strain (e.g. H3N2 and H5N1) for example.

The influenza virus antigen or antigenic preparation thereof may be produced by any of a number of commercially applicable processes, for example the split flu process described in patent no. DD 300 833 and DD 211 444, or WO2002097072 (equivalent to U.S. Pat. No. 7,316,813), all incorporated herein by reference.

The preparation process for a split vaccine may include a number of different filtration and/or other separation steps, such as ultracentrifugation, ultrafiltration, zonal centrifugation or chromatography (e.g. ion exchange) steps in a variety of combinations, and optionally an inactivation step e.g. with heat, formaldehyde or β-propiolactone or U.V.

which may be carried out before or after splitting. The splitting process may be carried out as a batch, continuous or semi-continuous process. A preferred splitting and purification process for a split immunogenic composition is described in WO2002097072.

In one embodiment, the influenza vaccine is prepared in the presence of low level of thiomersal, or in the absence of thiomersal. In another embodiment, the resulting influenza vaccine is stable in the absence of organomercurial preservatives, in particular the preparation contains no residual thiomersal. In particular the influenza virus preparation comprises a haemagglutinin antigen stabilised in the absence of thiomersal, or at low levels of thiomersal (generally 20 μg/ml or less, such as 15 μg/ml or less, 10 μg/ml or less, 5 μg/ml or less, or 2 μg/ml or less). Specifically the stabilization of B influenza strain is performed by a derivative of alpha tocopherol, such as alpha tocopherol succinate (also known as vitamin E succinate, i.e. VES). Such preparations and methods to prepare them are disclosed in WO 02/097072.

A preferred vaccine contains three inactivated split virion antigens prepared from the WHO recommended strains of the appropriate influenza season.

In one aspect the invention also relates to a method for isolating an influenza virus from a patient sample, comprising a step in which the patient sample is incubated with an avian cell, wherein the avian cell is growing in one of: a suspension culture, a serum-free medium, a protein-free medium, or any combination thereof, and to an influenza virus isolated by the said method.

In one aspect the invention relates to a process for preparing an influenza virus antigen for use in a vaccine, comprising steps of: (i) receiving an influenza virus that has never been propagated on an egg substrate; (ii) infecting a cell line with this influenza virus; and (iii) culturing the infected cells from step (ii) in order to produce an influenza virus, wherein the process uses an avian cell line for some or all steps.

Receiving in this context, as described below, may mean using or providing for use.

In one aspect the invention relates to a process for preparing an influenza virus antigen for use in a vaccine, comprising steps of: (i) receiving an influenza virus that has never been propagated on a substrate growing in a serum- containing medium; (ii) infecting a cell line with this influenza virus; and (iii) culturing the infected cells from step (ii) in order to produce influenza virus, wherein the process uses an avian cell line for some or all steps.

In one aspect the invention relates to a process for preparing an influenza virus antigen for use in a vaccine, comprising steps of: (i) receiving an influenza virus that was generated using reverse genetics techniques; (ii) infecting a cell line with this influenza virus; and (iii) culturing the infected cells from step (ii) in order to produce influenza virus, wherein the cell line is an avian cell line.

In one aspect the invention relates to a process for preparing a reassortant influenza virus, comprising steps of: (i) infecting a cell line with both a first strain of influenza virus having a first set of genome segments and a second strain of influenza virus having a second set of genome segments, wherein the first strain has a HA segment encoding a desired hemagglutinin; and (ii) culturing the infected cells from step (i) to produce influenza virus having at least one segment from the first set of genome segments and at least one segment from the second set of genome segments, provided that said at least one segment from the first set of genome segments includes the HA segment from the first strain, wherein the process uses an avian cell line for some or all steps.

### Medical Treatment and Delivery

In one aspect the invention relates to a method of inducing an immune response into a human subject, said method comprising administering to the subject the vaccine of the invention.

The vaccine of the invention may be administered by any suitable delivery route, such as intradermal, mucosal e.g. intranasal, oral specifically sub-lingual, intramuscular or subcutaneous. Other delivery routes are well known in the art.

The intramuscular delivery route may be suitable for adjuvanted influenza compositions.

The composition according to the invention may be presented in a monodose container, or alternatively, a multidose container, particularly suitable for a pandemic vaccine. In this instance an antimicrobial preservative such a thiomersal is typically present to prevent contamination during use. Thiomersal concentration may be at 25 μg/ 0.5 ml dose (i.e. 50 pg/mL). A thiomersal concentration of 5 μg/0.5 ml dose (i.e. 10 μg/ml) or 10 μg/0.5 ml dose (i.e. 20 μg/ml) is suitably present. A suitable IM delivery device could be used such as a needle-free liquid jet injection device, for example the Biojector 2000 (Bioject, Portland, Oreg.). Alternatively a pen-injector device, such as is used for at-home delivery of epinephrine, could be used to allow self administration of vaccine. The use of such delivery devices may be particularly amenable to large scale immunization campaigns such as would be required during a pandemic.

Intradermal delivery is another suitable route. Any suitable device may be used for intradermal delivery, for example a short needle device. Intradermal vaccines may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Also suitable are ballistic powder/particle delivery devices which use compressed gas to accelerate vaccine in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Suitably, a needle-free jet injector device is used. Suitably said device is pre-filled with the liquid vaccine formulation.

Alternatively the vaccine is administered intranasally. Typically, the vaccine is administered locally to the nasopharyngeal area, suitably without being inhaled into the lungs. It is desirable to use an intranasal delivery device which delivers the vaccine formulation to the nasopharyngeal area, without or substantially without it entering the lungs.

Suitable devices for intranasal administration of the vaccines according to the invention are spray devices. Suitable commercially available nasal spray devices include Accuspray™ (Becton Dickinson). Nebulisers produce a very fine spray which can be easily inhaled into the lungs and therefore does not efficiently reach the nasal mucosa. Nebulisers are therefore not preferred.

Suitable spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO91/13281 and EP311863B and EP516636, incorporated herein by reference. Such devices are commercially available from Pfeiffer GmbH and are also described in Bommer, R. Pharmaceutical Technology Europe, September 1999.

Suitable intranasal devices produce droplets (measured using water as the liquid) in the range 1 to 200 μm, suitably 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is a further suitable feature of an intranasal delivery system for use with the vaccines according to the invention. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device may be used for administering the vaccines according to the invention.

Alternatively, the transepidermal or transdermal or transcutaneous vaccination route is also contemplated in the present invention.

In one aspect of the invention the vaccination may be in a prime boost approach.

In one aspect of the present invention, the adjuvanted immunogenic composition for the first administration may be given intramuscularly, and the boosting composition, either adjuvanted or not, may be administered through a different route, for example transcutaneous, intradermal, subcutaneous, intranasal or sublingual.

In a specific embodiment, the composition for the first administration contains a HA amount of less than 15 μg for a pandemic influenza strain, and the boosting composition may contain a standard amount of 15 μg or, suitably a low amount of HA, i.e. below 15 μg, which, depending on the administration route, may be given in a smaller volume.

Although the vaccine of the invention may be administered as a single dose, components thereof may also be co-administered together at the same time or at different times (for instance influenza antigens could be administered separately, suitably at the same time as the administration of the adjuvant). In addition to a single route of administration, 2 different routes of administration may be used when two injections are administered. For example, the first administration (e.g. priming dose) of adjuvanted influenza antigens may be administered IM (or ID) and the second administration (e.g. booster dose) may be administered IN (or ID). In addition, the vaccines of the invention may be administered IM for priming doses and IN for booster doses.

The content of influenza antigens in the vaccine may be in the range 0.1-15 μg HA per influenza strain, suitably 1-10μg, most typically in the range 1-8 μg, in particular for a pandemic vaccine. A suitable content of influenza antigens will be less than or exactly 5 μg HA per influenza strain included in the vaccine. Following an initial vaccination, subjects may receive one or several booster immunizations adequately spaced.

### Influenza Strain

Suitably the influenza virus strain or strains to be included in the immunogenic or vaccine composition is/are seasonal strain(s), or strain(s) being associated with a pandemic outbreak or having the potential to be associated with a pandemic outbreak, or suitably, in a multivalent composition, a mixture of these strains.

Interpandemic strains are for example strains which circulate globally during seasonal periods such as but not limited to: H1N1, H1N2, H3N2 or B. Commercially available influenza vaccines are a trivalent combination including one influenza B strain and two influenza A strains (H1N1, H3N2).

The features of an influenza virus strain that give it the potential to cause a pandemic or an outbreak of influenza disease associated with pandemic influenza strains are: it contains a new haemagglutinin compared to the haemagglutinin in the currently circulating strains and therefore nearly all people are immunologically naïve or it contains a variation of a circulating strain to which the majority of people are immunologically naïve; it is capable of being transmitted horizontally in the human population; and it is pathogenic for humans. A new haemagglutinin may be one which has not been evident in the human population for an extended period of time, probably a number of decades, such as H2. Or it may be a haemagglutinin that has not been circulating in the human population before, for example H5, H9, H7 or H6 which are found in avian species (birds). In either case the majority, or at least a large proportion of, or even the entire population has not previously encountered the antigen and is immunologically naïve to it. At present, the influenza A virus that has been identified by the WHO as one that potentially could cause a pandemic in humans is the highly pathogenic H5N1 avian influenza virus. Therefore, the pandemic vaccine according to the invention will suitably comprise H5N1 virus. Three other suitable strains for inclusion into the claimed composition are H1N1, H9N2 or H7N1.

Certain parties are generally at an increased risk of becoming infected with influenza in a pandemic situation. The elderly, the chronically ill and small children are particularly susceptible but many young and apparently healthy people are also at risk. For H2 influenza, the part of the population born after 1968 is at an increased risk. It is important for these groups to be protected effectively as soon as possible and in a simple way. Another group of people who are at increased risk are travellers. People travel more today than ever before and the regions where most new viruses emerge, China and South East Asia, have become popular travel destinations in recent years. This change in travel patterns enables new viruses to reach around the globe in a matter of weeks rather than months or years.

Thus for these groups of people there is a particular need for vaccination to protect against influenza in a pandemic situation or a potential pandemic situation. Suitable strains are, but not limited to: H1N1, H5N1, H5N8, H5N9, H7N4, H9N2, H7N7, H7N3, H2N2 and H7N1. Other pandemic strains in human: H7N3 (2 cases reported in Canada), H10N7 (2 cases reported in Egypt) and H5N2 (1 case reported in Japan) and H7N2. An influenza strain which is a pandemic strain or a strain susceptible to be associated with a pandemics will be referred to in short in this document as a “pandemic strain”.

In one aspect the influenza virus is an influenza A or influenza B strain. In one aspect influenza A virus strain is of a H1, H3 or H5 hemagglutinin sub-type. The influenza virus may be a pandemic strain or a non pandemic strain.

In one aspect the strain is modified so as to be non lethal to avian cells.

Preferred influenza viruses of the invention (including seed viruses, viruses isolated from patient samples using avian cells, reassortant viruses, etc.) include hemagglutinin with a binding preference for oligosaccharides with a Sia([alpha]2,6)GaI terminal disaccharide compared to oligosaccharides with a Sia([alpha]2,3)GaI terminal disaccharide.

Preferred influenza viruses of the invention (including seed viruses, viruses isolated from patient samples using avian cells, reassortant viruses, etc.) include glycoproteins (including hemagglutinin) with a different glycosylation pattern than those obtained from egg-derived viruses. Thus the glycoproteins will comprise glycoforms that are not seen in viruses grown in chicken eggs e.g. they may have non-avian sugar linkages, including mammalian-like sugar linkages.

In one aspect of the invention, the human dose of the immunogenic composition contains an haemagglutinin (HA) from a single influenza strain, and is referred to as a “monovalent” influenza composition. In another aspect of the invention, the human dose of the immunogenic composition comprises haemagglutinin (HA) from more than one influenza strain, and is referred to as a “multivalent” influenza composition. A suitable multivalent composition according to the invention is a bivalent composition (comprising haemagglutinin (HA) from two influenza virus strains such as but not exclusively two strains associated to a pandemics or susceptible to be associated with a pandemic, e.g. H5=H2), a trivalent composition (comprising haemagglutinin (HA) from three influenza virus strains, optionally from two A strains, and one B strain such as but not limited to B/yamagata or B/Victoria), a quadrivalent composition (comprising haemagglutinin (HA) from four influenza virus strains) or a pentavalent composition (comprising haemagglutinin (HA) from five influenza virus strains). A suitable quadrivalent composition comprises haemagglutinin from two A strains and two B strains from different lineage (such as B/yamagata or B/Victoria). Alternatively a quadrivalent composition comprises haemagglutinin from three A strains (optionally H1N1, H3N2, and one A strain associated to a pandemic or susceptible to be associated to a pandemic) and one B strain (such as B/yamagata or B/Victoria). Another alternative quadrivalent composition comprises haemagglutinin from four A strains from a strain associated to a pandemic or susceptible to be associated to a pandemic, such as avian strains such as H5+H2+H7+H9. Specifically a multivalent adjuvanted pandemic composition such as a pandemic bi-valent (e.g. H5+H2) or trivalent or quadrivalent (e.g. H5+H2+H7+H9) offers the advantage of a pre-emptive immunisation against pandemic influenza A threats subtypes and durable priming against threat subtypes. Typically two doses are given from 6 weeks of age using a convenient schedule (e.g., 6-12 months apart), and optionally a periodic booster foreseen (e.g., 10 yrs). Optionally, such a pandemic vaccine may be combined with a seasonal vaccine.

A multivalent composition can also comprise more than 5 influenza strains such as 6, 7, 8, 9 or 10 influenza strains.

When two B strains are used in a multivalent seasonal composition, they can be from two different lineages (optionally from B/Victoria and B/Yamagata). At least one of said B strain, suitably both B strains, will be from a circulating lineage. Such a composition is particularly suitable for children. Suitably when the multivalent composition for use in children includes two B strains the quantity of antigen normally allotted to the B strain is divided among the two B strains. Specifically, the adjuvanted quadrivalent (H1+H3+both B lineages) influenza vaccine offers the advantage of enhanced prophylaxis for naïve children as its superior efficacy compared to unadjuvanted vaccines (in terms of both homologous and drift protection, and its efficacy against two circulating B lineages) and of possible year-round immunization based on age. One dose or two doses are suitably administered as early as from the age of 6 weeks, or between 6 to 35 months.

In a specific embodiment, the human dose of the immunogenic composition is a trivalent immunogenic or vaccine composition comprising haemagglutinin (HA) from two A strains (optionally H1N1, H3N2) and one B strain. Suitably the HA per strain is a low amount of HA (optionally 10 μg HA per strain or below) and is as defined above. Suitably the HA per strain is at about or below 5 μg, at about 2.5 μg or below. An adjuvant as defined herein may be included and in particular as defined in Table 1. Suitably the adjuvant composition is an oil-in-water emulsion comprising squalene, alpha-tocopherol, and polysorbate 80 at an amount of between 5-6 mg, between 5-6 mg and between 2-3 mg per dose, respectively. Alternatively, the adjuvant composition is an oil-in-water emulsion comprising squalene, alpha-tocopherol, and polysorbate 80 at an amount of between 2.5-3.5 mg, between 2-3 mg and between 1-2 mg per dose, respectively. These adjuvanted immunogenic compositions or vaccines are particularly suitable for the adult (18-60 years) or older children (3-17 years) population, and may provide cross-protection against H3N2 drift variants and against B strain from a different lineage.

In another specific embodiment, the human dose of the immunogenic composition is a quadrivalent immunogenic or vaccine composition comprising haemagglutinin (HA) from two A strains (optionally H1N1, H3N2) and two B strains (optionally from a different lineage, such as from B/Victoria and B/Yamagata). In another specific embodiment, the human dose of the immunogenic composition is a quadrivalent immunogenic or vaccine composition comprising haemagglutinin (HA) from two interpandemic A strains (optionally H1N1, H3N2), one B strain and one A strain associated to a pandemic or susceptible to be associated with a pandemic (optionally H5N1, H9N2, H7N7, H5N8, H5N9, H7N4, H7N3, H2N2, H10N7, H5N2, H7N2 and H7N1). In another specific embodiment, the human dose of the immunogenic composition is a quadrivalent immunogenic or vaccine composition comprising haemagglutinin (HA) from three interpandemic A strains (optionally H1N1, and two H3N2 strains) and one B strain. Suitably the HA per strain per dose is at about 15 μg. Suitably the HA per strain is a low amount of HA (optionally at about 10 μg HA per strain per dose or below, so as to achieve a maximum of 40-45 μg HA per dose of quadrivalent composition) and is as defined above. Suitably the HA per strain is at about or below 5 μg, at about 2.5 μg or below. An adjuvant where present may be any adjuvant as described herein.

In another specific embodiment, the human dose of the immunogenic composition is a pentavalent immunogenic or vaccine composition comprising haemagglutinin (HA) from two interpandemic A strains (optionally H1N1, H3N2), two B strains (optionally from a different lineage, such as from B/Victoria and B/Yamagata) and one A strain associated to a pandemic or susceptible to be associated with a pandemic (optionally H5N1, H9N2, H5N8, H5N9, H7N4, H7N7, H7N3, H2N2, H10N7, H5N2 and H7N1). In another specific embodiment, the human dose of the immunogenic composition is a pentavalent immunogenic or vaccine composition comprising haemagglutinin (HA) from three interpandemic A strains (optionally H1N1, and two H3N2 strains) and two B strains (optionally from a different lineage, such as from B/Victoria and B/Yamagata). Suitably the HA per strain is a low amount of HA (optionally 10 μg HA per strain or below) and is as defined above.

In one embodiment the multivalent compositions are adjuvanted, suitably with a squalene-based oil-in-water emulsion adjuvant. Accordingly in a specific embodiment the invention provides an influenza immunogenic composition comprising squalene and HA wherein the weight ratio squalene:total amount of HA (all influenza strains included) is in the range of between about 50-150 or about 150-400 (e.g. about 200-300). Such compositions are suitably but not exclusively for use in the elderly population and best balances reactogenicity and immunogenicity. In another embodiment, the invention provides an influenza immunogenic composition comprising squalene and HA wherein the weight ratio squalene:total amount of HA (all influenza strains included) is between about 50-400, e.g. about 50-100, 75-150, 75-200, 75-400, 100-200, 100-250 or 200-400. The ratio will suitably be such that at least two, suitably all three criteria (see Table 3 or 4 below) for protection will be met for a specific population. A TLR agonist, suitably a TLR-4 (e.g. chosen from: 3D-MPL, MPL; an AGP molecule such as RC527 or RC529, or ER804057) or TLR-9 (suitably a CpG oligonucleotide e.g. CpG7909) agonist may be included. Suitable weight ratios squalene:TLR-4 are of between about 100-450, e.g. 50-250, 50-150, 100-250, 200-250, 350-450. Suitable weight ratios squalene:TLR-9 are of between about 50-1000, e.g. 50-500, 100-1000, 100-400, 400-600. The HA can be from seasonal influenza strains. Such compositions are suitably but not exclusively for use in the adult or paediatric populations and best balance reactogenicity and immunogenicity. Suitably the HA is from at least three, at least four influenza strains. Suitably three seasonal (e.g. H1N1, H3N2, B) strains are present. Suitably when four strains are present they are from the group of: four seasonal strains (e.g. H1N1, H3N2, two B strains; or H1N1, B, two H3N2 strains) or the group of one pandemic (e.g. avian) strain plus three seasonal strains (e.g. H1N1, H3N2, B).

All the claimed adjuvanted immunogenic compositions or vaccine can advantageously rely on the adjuvant to provide a persistent immune response, over a period of time exceeding 6 months, suitably 12 months after the vaccination.

Suitably the above response(s), for example persistent immune response and those outlined in Table 3 or 4 is(are) obtained after one dose, or typically after two doses administered during the same on-going primary immune response. It is a particular advantage of the claimed composition that the immune response is obtained after only one dose of adjuvanted composition or vaccine. It is also suitable that two doses are administered during the same on-going primary immune response, suitably for naïve or immuno-compromised populations or individuals. Suitably, two doses might be needed in children, in particular below the age of 6 years or 9-10 years or in infants aged 0-3 years, not previously vaccinated.

Accordingly, there is provided in one aspect of the invention an immunogenic composition comprising a non-live pandemic influenza virus antigen preparation, in particular a split influenza virus or antigenic preparation thereof, for use in a one-dose or a two-doses vaccination against influenza, wherein the one-dose or the two-doses vaccination generates an immune response which meets at least one, suitably two or three, international regulatory requirements for influenza vaccines. In another particular embodiment said one-dose vaccination also or additionally generates a CD4 T cell immune response and/or a B cell memory response which is higher than that obtained with the non adjuvanted vaccine. In a particular embodiment said immune response is a cross-reactive antibody response or a cross-reactive CD4 T cell response or both. In a specific embodiment the human patient is immunologically naïve (i.e. does not have pre-existing immunity) to the vaccinating strain. In one aspect the vaccine composition contains a low HA antigen amount and an oil-in-water emulsion adjuvant with components at a level lower than those which had previously been thought useful, and which amounts are as defined herein. Specifically the vaccine composition is as defined herein. In particular the immunogenic properties of the vaccine composition are as defined herein. Suitably the vaccine is administered intramuscularly.

### Efficacy Criteria

The influenza medicament of the invention suitably meets certain international criteria for vaccines. Standards are applied internationally to measure the efficacy of influenza vaccines. Serological variables are assessed according to criteria of the European Agency for the Evaluation of Medicinal Products for human use (CHMP/BWP/214/96, Committee for Proprietary Medicinal Products (CPMP). Note for harmonization of requirements for influenza vaccines, 1997. CHMP/BWP/214/96 circular N° 96-0666:1-22) for clinical trials related to annual licensing procedures of influenza vaccines (Table 3 or Table 4). The requirements are different for adult populations (18-60 years) and elderly populations (>60 years) (Table 3). For interpandemic influenza vaccines, at least one of the assessments (seroconversion factor, seroconversion rate, seroprotection rate) should meet the European requirements, for all strains of influenza included in the vaccine. The proportion of titres equal or greater than 1:40 is regarded most relevant because these titres are expected to be the best correlate of protection [Beyer W et al. 1998. Clin Drug Invest; 15:1-12].

As specified in the “Guideline on dossier structure and content for pandemic influenza vaccine marketing authorisation application. (CHMP/VEG/4717/03, Apr. 5, 2004, or more recently EMEA/CHMP/VWP/263499/2006 of 24 Jan. 2007 entitled ‘Guidelines on flu vaccines prepared from viruses with a potential to cause a pandemic’, available on www.emea.eu.int), in the absence of specific criteria for influenza vaccines derived from non circulating strains, it is anticipated that a pandemic candidate vaccine should (at least) be able to elicit sufficient immunological responses to meet suitably all three of the current standards set for existing vaccines in unprimed adults or elderly subjects, after two doses of vaccine. The EMEA Guideline describes the situation that in case of a pandemic the population will be immunologically naive and therefore it is assumed that all three CHMP criteria for seasonal vaccines will be fulfilled by pandemic candidate vaccines. No explicit requirement to prove it in pre-vaccination seronegative subjects is required.

The compositions of the present invention suitably meet at least one such criteria for the strain included in the composition (one criteria is enough to obtain approval), suitably at least two, or typically at least all three criteria for protection as set forth in Table 3.

TABLE 3
(CHMP criteria)

| | 18-60 years | >60 years |
|---|---|---|
| Seroconversion rate* | >40% | >30% |
| Conversion factor** | >2.5 | >2.0 |
| Protection rate*** | >70% | >60% |

*Seroconversion rate is defined as the proportion of subjects in each group having a protective post-vaccination titre ≧1:40. The Seroconversion rate simply put is the % of subjects who have an HI titre before vaccination of <1:10 and ≧1:40 after vaccination. However, if the initial titre is ≧1:10 then there needs to be at least a fourfold increase in the amount of antibody after vaccination.
**Conversion factor is defined as the fold increase in serum HI geometric mean titres (GMTs) after vaccination, for each vaccine strain.
***Protection rate is defined as the proportion of subjects who were either seronegative prior to vaccination and have a (protective) post-vaccination HI titre of ≧1:40 or who were seropositive prior to vaccination and have a significant 4-fold increase in titre post-vaccination; it is normally accepted as indicating protection.

FDA uses slightly different age cut-off points, but their criteria are based on the CHMP criteria. Appropriate endpoints similarly include: 1) the percent of subjects achieving an HI antibody titer ≧1:40, and 2) rates of seroconversion, defined as a four-fold rise in HI antibody titer post-vaccination. The geometric mean titer (GMT) should be included in the results, but the data should include not only the point estimate, but also the lower bound of the 95% confidence interval of the incidence rate of seroconversion, and the day 42 incidence rate of HI titers ≧1:40 must exceed the target value. These data and the 95% confidence intervals (CI) of the point estimates of these evaluations should therefore be provided. FDA draft guidance requires that both targets be met. This is summarised in Table 4.

TABLE 4

| | 18-64 years | >64 years |
|---|---|---|
| Seroconversion rate * | >40% | >30% |
| Rate of HI titers ≧1:40 | >70% | >60% |

* The seroconversion rate is defined as: a) for subjects with a baseline titer ≧1:10, a 4-fold or greater rise; or b) for subjects with a baseline titer < 1:10, a rise to ≧1:40. These criteria must be met at the lower bound of the 95% CI for the true value.

In an alternative embodiment, the compositions of the present invention suitably meet at least one such criteria for the strain included in the composition, suitably both criteria for protection as set forth in Table 4.

Suitably this effect is achieved with a low dose of antigen, such as with 7.5 μg HA or even a lower antigen dose such as 3.8 μg or 1.9 μg of HA.

Suitably any or all of such criteria are also met for other populations, such as in children and in any immuno-compromised population.

Certain features of the invention include the following :

1. A process for preparing an influenza seed virus for vaccine manufacture, comprising steps of: (i) infecting a cell line with an influenza virus and (ii) amplifying the virus obtained from the infected cell line of (i) to produce influenza virus for use as a seed virus, wherein the cell line is an avian cell line.

2 A process for preparing an influenza seed virus for vaccine manufacture, comprising steps of: (i) infecting a cell line with an influenza virus; (ii) preparing a cDNA of at least one viral RNA segment of an influenza virus produced by the infected cell line obtained in step (i), and using the cDNA in a reverse genetics procedure to prepare a new influenza virus having at least one viral RNA segment in common with the influenza virus of step (i); and (iii) infecting a cell line with the new influenza virus, and (iv) amplifying the virus in order to produce a new influenza virus for use as a seed virus, wherein the cell line is an avian cell line for one or more of steps i, iii and iv.

3 A process according to claim 1 or 2 wherein the influenza virus of step (i) is obtained either directly from a patient or from a primary isolate in a cell line, or is a strain either circulating in the population or which has a hemagglutinin that is antigenically representative of an influenza virus that is circulating in the population.

4 A process according to claim 3 wherein the influenza virus is grown in a mammalian cell line before being infected into an avian cell line.

5 A process according to claim 4 wherein the mammalian cell line is an MDCK cell line.

6 A process according to any preceding claim wherein none of steps involves growth or passaging of influenza virus in eggs.

7 The process of any preceding claim, wherein the avian cell line is a continuous diploid avian cell line.

8 The process of any preceding claim, wherein the avian cell line is a duck cell line.

9 The process of any preceding claim, wherein the duck cell line is as manufactured in, or as otherwise disclosed in, WO2008129058 or WO2003076601.

10 The process of claim 9 wherein the cell line is a stem cell line, for example EB66.

11 The process of any preceding claim wherein the influenza virus is infected into a non-human cell line, such as an MDCK cell line, after use of the avian cell line.

12 The process of claims 1-3, 6-10 wherein avian cells are the only cell culture cells used in the production of a seed vaccine.

13 A process for preparing an influenza virus vaccine, wherein the seed virus according to any preceding claim is cultured to produce influenza virus for vaccine manufacture.

14 A process according to claim 13 wherein the virus is treated to produce a vaccine.

15 A process for making a multivalent influenza vaccine, comprising performing the process of claim 14 for a an individual influenza virus strain, and mixing that strain with one or more other influenza vaccines, optionally made using the process of claim 13, to make a multi-valent influenza vaccine.

16 A vaccine manufactured according to claim 14 or 15.

17 A method of treating an individual in need thereof, the method comprising vaccinating that individual using a vaccine according to claim 16.

### General Language

The teaching of all references in the present application, including patent applications and granted patents, are herein fully incorporated by reference. Any patent application to which this application claims priority is incorporated by reference herein in its entirety in the manner described herein for publications and references.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Embodiments herein relating to “vaccine compositions” of the invention are also applicable to embodiments relating to “immunogenic compositions” of the invention, and vice versa. The term “about” (or “around”) in all numerical values allows for a 5% variation, i.e. a value of about 1.25% would mean from between 1.19%-1.31%.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The invention will be further described by reference to the following, non-limiting, examples:

### EXAMPLES

### I. Isolation and Amplification of Virus

As soon as clinical isolate (usually kept at 4° C. or −70° C.), is received, the sample is thawed. A part or the total of the sample is diluted in cell culture medium. The residual part of the sample can be frozen.

The sample diluted in culture medium, typically EBx-PROI medium from SAFC or ultraMDCK from Lonza, is added to cell culture of avian cells such as EB66 or MDCK cells as appropriate, based on a final volume/volume ratio ranging from 1:10 to 10-8:1. In particular, temperature is set between 30 and 37° C., preferably 33-35° C. Cell density can vary from 1×10⁵ to 5×10⁶ cells/ml. Trypsin or trypsin-like proteases from animal, bacterial or plant origin can be used. Trypsin concentration is described in Lenette et al (1995). The preferred method is to seed cells with the clinical isolate diluted in EBx-PROI medium in multiwell plates with various volumes of clinical isolate or in flasks if sample can be highly diluted (same method as for amplification see below).

After several days of viral amplification, typically 2 to 7 days, the cytopathic effect (CPE) is observed. The virus is harvested when the CPE appears quite complete and the harvest can constitute a virus seed, stored at −70° C.

The cell substrate is preferably a continuous avian cell line and/or a duck cell line or a cell line with a non tumorigenic phenotype.

In the case where the viral amplification has occurred in another animal cell line, for example, MDCK cell line, virus resulting from this amplification is considered as the virus sample, and the virus amplification is performed as described here above.

In case reverse genetics is used to prepare the influenza strain (FIG. 2), the rescue virus can either be amplified either on egg or on a cell line, but preferably a cell line. The method described here can be applied to both types of seeds when expanded on e.g. an EB66 cell line for new seed preparation or vaccine preparation itself.

The following parameters may be optimised by the skilled person using conventional techniques:
duration of the pre-culture before the day of infection: 2 or 3 days,
temperature: 33 or 35° C.,
nature of recombinant trypsin (Trypzean or TrypLE) in a single addition or each day during viral replication
MOI: 10⁻³ to 10⁻⁶,
The harvest at day 3, day 4 or day 5 post-infection.

### II: Testing of Cell Lines to Determine if the Sequence of HA & NA of EB66 Cell Culture Amplified Influenza Virus is Altered

The EB66 cell line was tested to determine if there was an effect on HA sequence of the influenza virus grown within the EB66 cell line.

### II.1. Isolation and Amplification of Pandemic Virus Strain

The principle of the method was to test on EB66 cell substrate a dilution range of the selected seed of a given generation in order to produce multiple candidate-seeds for the next generation, the best of which was selected. This method requires knowledge of the titer of the initial seed, named P0.

In detail, a PO seed received from CDC was used. This P0 seed is an H5N1 pandemic strain first isolated in Indonesia from an infected person and further processed into a reassortant virus termed Indonesia/05/2005(H5N1)/PR8-IBCDC-RG2 containing the 6 internal virion protein coding genes from the low pathogenic H1N1 A/Puerto-Rico/8/34 strain. The reassortant virus, hereinafter referred to as A/Indonesia/05/2005, was recovered by transfection of plasmid DNA into Vero cells. The reassortant strain was supplied by the CDC, USA (human isolate, 8.37 log CCID₅₀/ml) after two passages on eggs.

For the EB66-derived antigen, the PO seed was used to infect EB66 cells around 1-3 10⁶ cells/ml. This infection was performed based on a MOI (Multiplicity of Infection, typically from 10⁻³ to 10⁻⁶) using TrypLE (0.5-2.5 mrPU/10⁶ cells/ml) added at time of infetion. The resulting P1 seed was harvested and frozen 3 days later; a sample was thawed to initiate the titration of its virus load according to the Reed and Muench method (Am.J;Hyg.1938, 27: 493-497). The titers then obtained were ranked and the best harvest was used as P1 seeds to inoculate EB66 cells in order to prepare P2 seeds. The P2 and P3 seeds were prepared according to the same protocol.

For the egg-derived antigen, the same seed was amplified in the seasonal “Fluarix” egg process as outlined in, for example, WO2002097072.

### Results

Using the protocol of amplification of EB66 cell line, HA and NA genes of P0 and P3 seed were sequenced. No nucleotide change was noticed. This indicates that EB66 is a suitable cell line to keep the native HA sequence as found in the clinical isolate. Further experiments were carried out as in Example III.

### II.2. Isolation and Amplification of Seasonal Influenza Virus Strains

### II.2.1 Introduction

Potential mutation events that could arise during EB66 cells viral adaptation were further investigated. Sequencing experiments were performed on HA and NA genes of seasonal strains before and after culture on EB66 cells and compare to egg or MDCK based amplification.

### II.2.2. Material and Methods

Sample identifications and number of passages on eggs or EB66 cells are shown in table 5. E4E2 means 6 passages on eggs prior EB66 culture. P0 and P3 means before and after 3 passages on EB66 cells or MDCK, respectively:

TABLE 5

| | Number of passages on Eggs |
|---|---|
| Sample identifications | or on EB66 or on MDCK |
| B/Malaysia/2506/20004 | P0 (=E4E2) |
| B/Malaysia/2506/20004 | P3 |
| A/Solomon lslands/03/2006(H1N1) | P0 (=E2E1) |
| A/Solomon Islands/03/2006(H1N1) | P3 |
| A/Wisconsin/67/2005(H3N2) | P0 (=E4E2) |
| A/Wisconsin/67/2005(H3N2) | P1 |
| A/Wisconsin/67/2005(H3N2) | P2 |
| A/Wisconsin/67/2005(H3N2) | P3 |
| A/Wisconsin/67/2005(H3N2) | P4 |

Viruses were amplified based on same principle described in §11.1. In detail, a P0 seed received from CDC or NIBSC was used to infect EB66 cells around 2.5-5 10⁶ cells/ml. This infection was performed based on a MOI (Multiplicity of Infection, typically from 10⁻³ to 10⁻⁶) using Trypzean with concentration from 0.3 to 0.8 USP/10⁶ cells/ml added daily. When MDCK was used as cell substrate, cells were seeded at 15,000 cells/cm². After 3 days, the infection was performed based on diluted volume from 10⁻² to 10⁻⁷ and recombinant trypsin, tryp-LE, was added at a concentration of 3 mrPU/ml at the time of infection and at day 1 post-infection . The resulting P1 seed was harvested and frozen 3 days later; a sample was thawed at the same date to initiate the titration of its virus load according to the Reed and Muench method (Am.J;Hyg.1938, 27: 493-497). The titers then obtained were ranked and the two best harvests were immediately used as P1 seeds to inoculate EB66 cells in order to prepare two P2 seeds. The P2 and P3 seeds were prepared according to the same protocol.

### Nucleic Acid Extractions and Reverse Transcriptase Reactions:

RNAs were extracted from 200 μl of sample using the High Pure Viral Nucleic Acid kit (Roche) following the manufacturer's instructions. Extracted RNAs were reverse transcribed to cDNA using Superscript III enzyme (Invitrogen) following the manufacturer's instructions. The RT reaction was carried out with an initial denaturation at 65° C. for 5 min, followed by 5 min at 25° C., 1 h at 50° C., and 15 min at 70° C. Each gene was full-length amplified, by two independent PCR reactions using the specific primers described in Table 6.

TABLE 6
(Sequences of primers used to amplify HA and NA genes)

| | | Gene | |
|---|---|---|---|
| Strains | Primers | Size (bp) | 5′-3′ Sequence |
| Flu B | HaB-F1 | 1757 | GAAGCAGAGCATTTTCTAATATCCACAA |
| | HaB-R1 | | ACAAGCACTACAATAAAGGAAAATACAGGG |
| | NaB-F1 | 1401 | GAAGCAAATAGGCCAAAAATGAACA |
| | NaB-R1 | | TTAGAACAGACTCAACCATTCCTCCA |
| H1N1 | H1-F1 | 1698 | AAGCAGGGGAAAATAAAAACAACCAAA |
| | H1-R1 | | AAGGGTGTTTTTTCTTATATTTCTGAA |
| | N1-F1 | 1413 | AGCAAAAGCAGGAGTTTA |
| | N1-R1 | | AACAAGGAGTTTTTTCAACGAA |
| H3N2 | H3-F1 | 1701 | GCAAAAGCAGGGGATAATTCTATTAACC |
| | H3-R1 | | AGTAGAAACAAGGGTGTTTTTAATTAATG |
| | N2-F1 | 1410 | AGCAAAAGCAGGAGTGAAGAT |
| | N2-R1 | | ACAAGGAGTTTTTTTCTAAAATTGCGAA |

The Ha and Na genes of samples at P3 were amplified by two independent PCR reactions. The first one uses the Platinium HiFi enzyme and the other one uses the Takara Ex Taq enzyme. For amplification using Platinium HiFi, the cycling conditions consisted in 1 cycle of 95° C. for 2 min followed by 35 cycles of 95° C. for 30 sec, 55° C. for 30 sec, and 68° C. for 2 min. For amplification using Takara Ex Taq, the cycling conditions consisted in 1 cycle of 95° C. for 2 min followed by 35 cycles of 95° C. for 30 sec, 55° C. for 30 sec, and 72° C. for 2 min. Due to amplification failures when using annealing temperature of 55° C., the annealing temperature that was used for the N1 gene was 42° C. and 45° C. for PO and P3, respectively. Each amplification was carried out in a final volume of 50 μl. 5 μl of the amplicons were then mixed with 1.25 μl of loading buffer and run on a 1.5% agarose gel. After electrophoresis, the gel was stained with Sybrsafe. Since, in this first set of experiments, the amplification with the Ex Taq enzyme was more efficient than with the Platinium HiFi, the amplification performed later were exclusively performed by using the Ex Taq enzyme.

PCR amplicons were purified using the ExoSap-IT enzyme (GE Healthcare) following the manufacturer's instructions. Purified amplicons were then sequenced on each strand with the sequencing primers depicted in the file attached below, using the ABI PRISM Big Dye Terminator cycle sequencing kit (Applied Biosystems), and an AB13730 sequencer. The forward and reverse sequences were aligned and trimmed to obtain a fragment that corresponds to the complete sequence.

TABLE 7

| Strains | Primers | 5′-3′ Sequence |
|---|---|---|
| H1N1 | H1-F1 | 5′-AAGCAGGGGAAAATAAAAACAACCAAA-3′ |
| | H1-R1 | 5′-AAG GGTGTTTTTTCTTATATTTCTGAA-3′ |
| | H1-F-seq3 | 5′-AACAAATTGGAAAGAAGGATGGAAAACT-3′ |
| | H1-R3-Serge | 5′-TTGGGCCATGAGCTTTCTTTG-3′ |
| | H1-F5-Serge | 5′-GCTTATGTCTCTGTAGTGTCTTCA-3′ |
| | H1-R5-Serge | 5′-AATGAAACCGGCAATGGCTC-3′ |
| | N1-F1 | 5′-AGCAAAAGCAGGAGTTTA-3′ |
| | N1-R1 | 5′-AACAAGGAGTTTTTTCAACGAA-3′ |
| | N1-R-seq2 | 5′-CTGGACCAGAAATTCCGATTGTTA-3′ |
| | N1-F-seq3 | 5′-GATTGGTCAGGGTACAGCGGAAGT-3′ |
| | N1-R-seq1 | 5′-GGTCAGGGTACAGCGGAAGTTTTG-3′ |
| H3N2 | H3-F 1 | 5′-GCAAAAGCAGGGGATAATTCTATTAACC-3′ |
| | H3-R1 | 5′-AGTAGAAACAAGGGTGTTTTTAATTAATG-3′ |
| | H3-F-seq3 | 5′-CGGAGCTTCTTGTTGCCCTGGAG-3′ |
| | H3-R1-Serge | 5′-CAAATTTTTCATTGTTTGGCATAGTCA-3′ |
| | H3-F8-Serge | 5′-CTTTAGTAGATTGAATTGGTT-3′ |
| | H3-R8-Serge | 5′-CTCAGAATTTTGATGCCTG-3′ |
| | N2-F1 | 5′-AGCAAAAGCAGGAGTGAAGAT-3′ |
| | N2-R1 | 5′-ACAAGGAGTTTTTTTCTAAAATTGCGAA-3′ |
| | N2-F-seq3 | 5′-ATGGGAAGAACGATCAGCGAGAA-3′ |
| | N2-R-seq3 | 5′-CCCACCAGCGGAAAGCCTAATC-3′ |
| Flu B | HaB-F1 | 5′-GAAGCAGAGCATTTTCTAATATCCACAA-3′ |
| | HaB-R1 | 5′-ACAAG CACTACAATAAAGGAAAATACAGGG-3′ |
| | HaB-F-seq3 | 5′-AGCACTCAAGAGGCCATAAACAAGATAA-3′ |
| | HaB-R-seq3 | 5′-GGGTCCTCCTGGTGCATTTTCTG-3′ |
| | BHa-F8-Serge | 5′-TTCGCAACAATGGCTTGG-3′ |
| | BHa-RinfB2- | 5′-CCAGCAATAGCTCCGAAGAAA-3′ |
| | Serge | |
| | NaB-F1 | 5′-GAAGCAAATAGGCCAAAAATGAACA-3′ |
| | NaB-R1 | 5′-TTAGAACAGACTCAACCATTCCTCCA-3′ |
| | NaB-F-seq3 | 5′-CCGCATGCCATGATGGTAAGGAA-3′ |
| | NaB-F-seq4 | 5′-GGGGATGGGGCTGTATGTCAAGT-3′ |
| | NaB-R-seq3 | 5′-TTGTTTCTGTCTCCTCTTGTTCCATTGT-3′ |

### II.2.3. Results

The sequences obtained from the tested samples (P0 and P3) were aligned and compared to the respective seasonal strain sequences available within the databases.

### II.2.3.1. FluB HA & NA

At position 633 of the FluB HA, a double population (R) was observed at P0 and P3 showing no sequence evolution between both passages. At position 641, a double population was observed (Y) at P0 and P3 that was expected from the sequences collected from the databases. Taken together, these results demonstrated the absence of impact of culturing the FluB seasonal strain on EB66 cells on the FluB HA gene.

Likewise, the alignments also demonstrated a 100% sequence homology between the NA FluB sequence before and after passages on EB66 cells.

The alignments results also demonstrated a 100% sequence homology between the H1 FluA sequence before and after passages on EB66 cells.

The alignments also demonstrated a 100% sequence homology between the N1 FluA sequence before and after passages on EB66 cells.

### II.2.3.2. FluA HA & NA

The FluA H3 gene sequence alignments were analysed at P0, P1, P2 and P3. No difference was observed between sequences observed at P1 and P2 when compared with the P0 starting sequence. However a double population (at position 522) (A/T) was observed in the sequence of P3. The alignment of P4 demonstrated that at the same position a unique population was detected (T). This mutation is not silent since a Lysine (AAA) was replaced by an Asparagine (AAT).

The alignments results demonstrated a 100% sequence homology between the N2 FluA sequence before and after passages on EB66 cells.

### II.2.4 Summary

Sequencing experiments were performed on the HA and NA genes of two seasonal Flu strains (B/Malaysia/2506/20004; A/Solomon Islands/03/2006(H1N1)) before and after three passages on EB66 cells. The results showed a 100% sequence homology between the HA and NA sequences after 3 passages on EB66 cells when compared to the reference sequence (P0).

The N2 sequence of the A/Wisconsin/67/2005(H3N2) strain also showed 100% sequence homology between NA sequences after 3 passages on EB66 cells when compared to the reference sequence (P0). The one exception was discovered in the HA gene of strain A/Wisconsin/67/2005(H3N2) where 1 mutation at position 522 (detection of both A and T) was observed at passage P3. Additional sequencing data collected at passage 4 demonstrated that the mutated nucleotide (T) replaced totally the original sequence (A). This mutation is a non silent mutation since an amino acid change is expected (Lysine replaced by Asparagine)

### III. Testing of Influenza Strains for Virus Immunogenicity

Sequencing of hemagglutinin from strains can be performed after each passage, and if significant mutation(s) is (are) identified in egg-derived seeds, but not in the cell-derived isolates, preclinical in vivo experiments are usually performed.

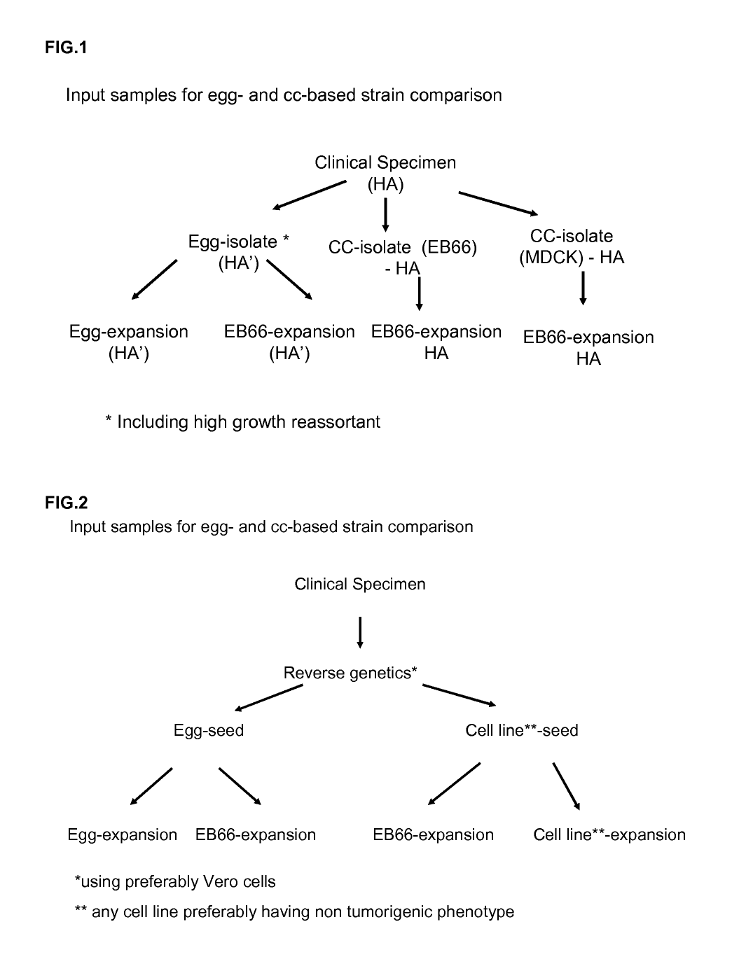

Suitable approaches for comparing egg and cell culture processes are shown in FIGS. 1 and 2.

The experiments below can be done in a stepwise fashion to determine how the cell-based viral isolates or vaccines's antigenicity (e.g. bi SRD testing) or immunogenicity potential compares to egg-derived viral seeds or purified inactivated virus:

1. Intranasal inoculation of naive ferrets with live seeds derived from avian cell line, eggs or MDCK cells to compare the quality of both homologous and heterologous antibody responses induced by the virus.
2. Comparative immunogenicity of both homologous and heterologous antibody responses in naïve or primed mice using purified inactivated virus such as split virus, with or without adjuvant.
3. Comparative immunogenicity and protection to both homologous and heterologous strains in naïve ferrets using purified, inactivated virus.

### Analytical Methods

### Serum Hemagglutination Inhibition Assay (HI)

The principle of the HI test is based on the ability of specific anti-influenza antibodies to inhibit hemagglutination of chicken or horse red blood cells (RBC) by influenza virus hemagglutinin (HA).

In this assay, prior to determination of HA inhibiting activity, serum is heated at 56 degrees C. to inactivate complement. Elimination of non-specific agglutination is achieved by treating sera with receptor destroying enzyme (RDE). To 50 μl of serum is added 200 μl of RDE (100 units/ml) for 12 to 18 hours at 37 degree C. 150 μl of sodium citrate (2.5%) is added for 30 min at 56 degree C. to inactivate the RDE. The sample volume is made up to 500 μl with PBS (to give a final sample dilution of 1:100). Two-fold serial dilution of each sample are tested for their ability to inhibit the agglutination of 0.5% chicken red blood cells or 1% horse red blood cells by whole/split Influenza virus homologous to the seed used for production of antigens and heterosubtypic strains in a standard HAI assay.

### Neutralising Antibody Assay

Virus neutralization by antibodies contained in the serum can be determined in a neutralization assay. Sera are used in the assay without further treatment. In this assay a standardized amount of virus is mixed with serial dilutions of serum and incubated to allow binding of the antibodies to the virus. A cell suspension, containing a defined amount of MDCK cells is then added to the mixture of virus and antiserum and incubated at 35° C. during 5 to 7 days. After the incubation period, virus replication is scored directly or visualized by hemagglutination of chicken red blood cells. The 50% neutralization titer of a serum is calculated by the method of Reed and Muench (Am.J;Hyg.1938, 27: 493-497).

### Viral Titration in Nasal Washes

In this assay all nasal samples are first sterile filtered through Spin X filters (Costar) to remove any bacterial contamination. 50 μl of serial ten-fold dilutions of nasal washes are transferred to microtiter plates containing 50 μl of medium (10 wells/dilution). 100 μl of MDCK cells (2.4×10⁵ cells/ml) are then added to each well and incubated at 35° C. for 5-7 days.

After 6-7 days of incubation, the culture medium is gently removed and 100 μl of a 1/15 WST-1 containing medium is added and incubated for another 18 hrs.

The intensity of the yellow formazan dye produced upon reduction of WST-1 by viable cells is proportional to the number of viable cells present in the well at the end of the viral titration assay and is quantified by measuring the absorbance of each well at the appropriate wavelength (450 nanometers). The cut-off is defined as the OD average of uninfected control cells —0.3 OD (0.3 OD correspond to +/−3 StDev of OD of uninfected control cells). A positive score is defined when OD is <cut-off and in contrast a negative score is defined when OD is >cut-off. Viral shedding titers were determined by “Reed and Muench (reference)” and expressed as Log TCID50/ml.

### III.1. Pandemic Strain

### III.1.1.1. Intramuscular Inoculation of Ferrets

Influenza infection in the ferret model closely mimics human influenza, with regards to the sensitivity and the clinical symptoms. The ferret is extremely sensitive to infection with human influenza viruses without prior adaptation of viral strains. Therefore, it provides an excellent model system for studies of protection conferred by administered influenza vaccines.

In addition, post infection ferret antisera are important tools to evaluate antigenic drifts and compare antigenicity of subtypic strains.

The study assesses the potential benefit of EB66 derived seeds to induce at least similar level of neutralizing or hemagglutination inhibition antibodies (compared to Embryonated egg derived seeds).

Female ferrets (Mustela putorius furo) (3-5 ferrets/group) aged 14 -20 weeks were inoculated twice via the intramuscular route with either cell derived seeds or embryonated egg derived seed (dose range from 1.9 to 15 μg). Alternatively a dose range of each inoculum i.e. 10-fold dilution steps could be inoculated to allow a more sensitive comparison if needed.

To assess antibody responses, serum samples may be collected from animals before immunization and up to 21 days after each intramuscular administration. Serum samples can be tested for their hemagglutination inhibition and neutralizing activities against the homotypic influenza strain.

The present experiment was carried out using an H5N1 pandemic strain obtained as described in §11.1 herein above. Groups of five ferrets were immunized twice intramuscularly, 21 days apart, with the monovalent A/Indonesia/05/2005 H5N1 split antigen produced either in EB66 cell culcture or in eggs. Both the cell culture and egg-based formulations, adjuvanted with the AS03 Adjuvant system and unadjuvanted, were compared. The dose and volume (500 μl) were similar to the human dose anticipated for the phase I clinical trial. Seven groups were defined as follows

Group 1: 15 μg HA EB66-based antigen (n=5)

Group 2: 3.8 μg HA EB66-based antigen/ASO3A (n=5)

Group 3: 1.9 μg HA EB66-based antigen/ASO3B (n=5)

Group 4: 15 μg HA egg-based antigen (n=5)

Group 5: 3.8 μg HA egg-based antigen/ASO3A (n=5)

Group 6: 1.9 μg HA egg-based antigen/ASO3B (n=5)

Group 7: Saline (n=3)

The AS03 adjuvant system used in this experiment as well as in the experiments described below is an oil-in-water emulsion-based adjuvant system comprising DL-alpha-tocopherol, squalene and polysorbate 80. Its preparation is described in WO 2008/043774 (example II therein). For the purpose of the present invention, a dose of AS03A contains 11.86 mg alpha-tocopherol, and AS03B consists of half a dose of AS03A (5.93 mg alpha-tocopherol). Thus a full human dose was injected in the ferrets.1/10 of a human dose was injected into mice, to maintain the same ratio of antigen/adjuvant as in a human dose.

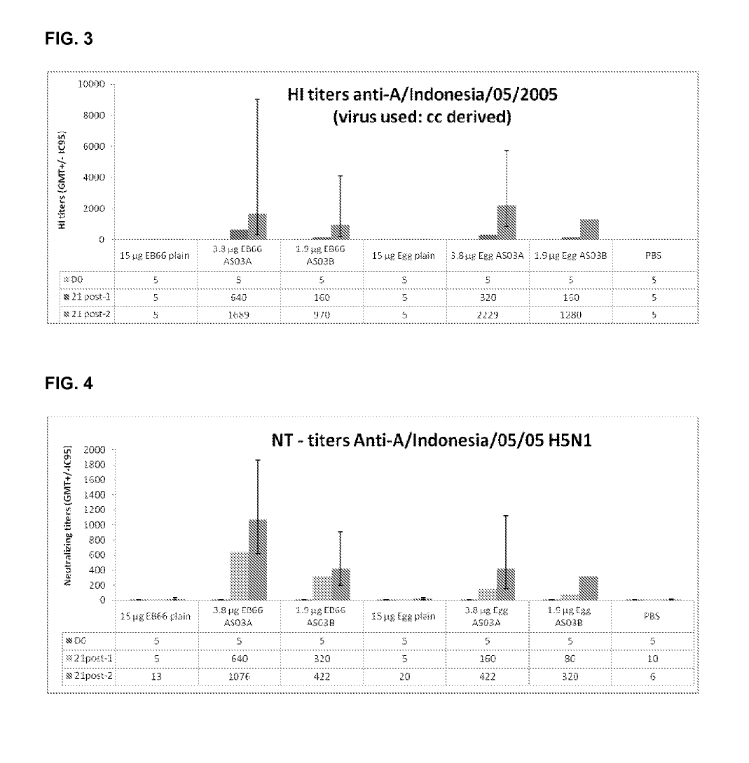

The humoral immune response to the different formulations was measured at day 0 (pool of 5 ferrets/group), 21 days after first immunization (post-1, pool of 5 ferrets/group) and 21 days after second immunization (post-2, individually) by HI and NT assays. EB66-based split antigen was used as the reagent for the HI assay. The data are presented in Table 8/FIG. 3 (HI titers) and Table 9/FIG. 4 (NT titers).

TABLE 8
HI titers anti-A/Indonesia/05/2005 in ferrets

| group | Day 0 | post-1 | post-2 |
|---|---|---|---|
| 1 | 5 | 5 | 5 |
| 2 | 5 | 640 | 1689 |
| 3 | 5 | 160 | 970 |
| 4 | 5 | 5 | 5 |
| 5 | 5 | 320 | 2229 |
| 6 | 5 | 160 | 1280 |
| 7 | 5 | 5 | 5 |

TABLE 9
NT titers anti-A/Indonesia/05/2005 in ferrets

| group | Day 0 | post-1 | post-2 |
|---|---|---|---|
| 1 | 5 | 5 | 13 |
| 2 | 5 | 640 | 1076 |
| 3 | 5 | 320 | 422 |
| 4 | 5 | 5 | 20 |
| 5 | 5 | 160 | 422 |
| 6 | 5 | 80 | 320 |
| 7 | 5 | 10 | 6 |

In conclusion, this preliminary immunogenicity study in ferrets showed the EB66 cell culture derived pandemic H5N1 vaccine candidate is able to induce an immune response in ferrets, similar to egg-based H5N1 vaccine.

III.1.2. Comparative Immunogenicity in Naïve Mice

C57BL/C6 mice are immunized intramuscularly or intraperitoneal on days 0 and 21 with 50 μl of optionally adjuvanted antigens derived either from EB66 or embryonated egg derived purified inactivated virus. Animals are bled 21 days after the first immunization and 14 days after the second administration via the saphenous vein or by cardiac puncture. Serum samples may be tested for their hemagglutination inhibition and neutralizing activities against the homotypic and/or a panel of heterosubtypic influenza strains to compare their ability to induce cross-reactive antibody responses. EB66 derived seeds is expected to induce at least similar cross-reactive antibody responses compared to embryonated egg derived seed.

For the present experiment, groups of female C57BL/6 mice, six weeks old, received two intramuscular injections (50 μl each), 21 days apart with monovalent A/Indonesia/05/2005 H5N1 split antigen produced either in EB66 cell culture or in eggs as described for §11.1. hereinabove. The groups were designed in order to test the bioequivalence (limit range 0.5-2%). Five groups were defined as follows:

Group 8: (20 mice) 3.8 μg EB66-based antigen/AS03A

Group 9: (30 mice) 1.9 μg EB66-based antigen/AS03B

Group 10: (20 mice) 3.8 μg Egg-based antigen/AS03A

Group 11: (30 mice) 1.9 μg Egg-based antigen/AS03B

Group 12: (14 mice) Saline

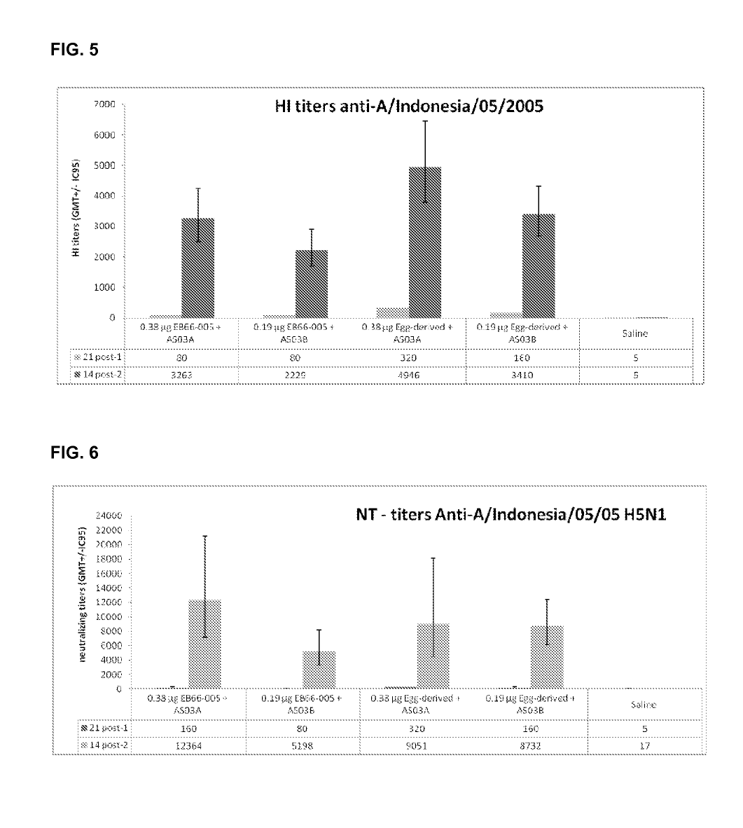

AS03A and ASO3B are as defined herein above. Serum samples were collected 21 days after the first immunization and 14 days after the second immunization. Specific anti-A/Indonesia/05/2005 H5N1 HI titers and NT titers were measured in one pool per group for post dose 1 samples and individually for post dose 2 samples. The data are presented in Table10/FIG. 5 (HI titers) and Table11/FIG. 6 (NT titers).

TABLE 10
HI titers anti-A/Indonesia/05/2005 in mice

| group | Post-1 | Post-2 | Seroconversion rate (%) |
|---|---|---|---|
| 8 | 80 | 3263 | 100 |
| 9 | 80 | 2229 | 97 |
| 10 | 320 | 4946 | 95 |
| 11 | 160 | 3410 | 93 |
| 12 | 5 | 5 | 0 |

Strong HI titers were induced in all adjuvanted vaccine groups. HI titers obtained with the EB66 cell culture antigen were lower than those obtained with the egg-based antigen but the difference between antigens was less than 2 fold. Considered within the inherent variability of the in vivo experimentations, a bioequivalence was detected between EB66 cell culture and egg-derived antigens.

TABLE 11
NT titers anti-A/Indonesia/05/2005 in mice

| group | Post-1 | Post-2 | Seroconversion rate (%) |
|---|---|---|---|
| 8 | 160 | 12364 | 100 |
| 9 | 80 | 5198 | 100 |
| 10 | 320 | 9051 | 95 |
| 11 | 160 | 8732 | 97 |
| 12 | 5 | 17 | 0 |

High NT titers were induced in all adjuvanted groups. A bioequivalence was detected between EB66 cell culture and egg-derived antigens.

Serum samples were also tested for their neutralizing activities against a heterosubtypic influenza strain, namely strain A/Vietnam/1194/2004 to determine the cross-reactive antibody response. The data are presented in table 12. Seroconversion takes place when the titer post-immunization is four times higher than pre-immunization.

TABLE 12
NT titers anti-A/Vietnam/1194/2004 in mice

| group | Post-1 | Post-2 | Seroconversion |
|---|---|---|---|
| 1 | 20 | 127 | 60% |
| 2 | 20 | 66 | 27% |
| 3 | 10 | 93 | 50% |
| 4 | 20 | 73 | 38% |

A cross-response was detected: anti-H5N1 A/Vietnam/1194/2004 NT titers were detected after immunization with H5N1 A/Indonesia/05/2005.

III.1.3. Ferret Challenge Experiment

The objective of this experiment is to compare the immunogenicity and the efficacy of EB66 cell culture derived and egg derived formulations using homologous wild-type A/H5N1/Indonesia/05/2005 virus challenge.

Female ferrets (Mustela putorius furo) (6 ferrets/group) aged about 12 months are injected with two intramuscular immunizations, 21 days apart with 500 μl of purified (e.g. split or sub-unit) antigens derived from either EB66 or embryonated egg derived isolates. 28 days after the second immunization, ferrets are challenged by the intratracheal route with 10⁵ Log CCID50 of homotypic influenza strain. Nasal washes are collected at day 1 before and up to 5 days after challenge to measure viral replication. Body temperature is continuously monitored. Serum samples are collected at day 0, day 21 (post 1^{st} immunization), 21 and 27 days after the second immunization to measure neutralizing and hemagglutinin inhibition antibody titers against homotypic and heterosubtypic strains.

III.2. Seasonal Strains

Cell culture derived saisonal Flu strains were evaluated in a ferret challenge experiment. The objective of this experiment is to compare the immunogenicity and the efficacy of EB66 cell culture derived, MDCK cell culture derived or egg derived formulations, as well as to compare cross-reactive antibody responses between EB66-, MDCK- and egg-derived seeds.

Intranasal challenge was performed on female ferrets (Mustela putorius furo) (4 ferrets/group) aged 14 -20 weeks with 250 μl of purified, 5 to 7 Log TCID50/ml derived from either MDCK, EB66 or embryonated egg derived isolates. Viral strains used are as described for §11.2 and listed in Table 5 above. Hence, ten groups are defined as follows (E3 means 3 passages on eggs, E6 means 6 passages on eggs, P0 means zero passages on cells, and P3 means 3 passages on cells):

| group | Flu type | isolate | sample preparation |
|---|---|---|---|
| 13 | H1N1 | WT A/Solomon Islands/03/ | Egg (E3-P0) |
| | | 2006 | |
| 14 | H1N1 | WT A/Solomon Islands/03/ | Egg → MDCK (E3-P3) |
| | | 2006 | |
| 15 | H1N1 | WT A/Solomon Islands/03/ | Egg → EB66 (E3-P3) |
| | | 2006 | |
| 16 | H3N2 | WT A/Wisconsin/67/2005 | Egg (E6-P0) |
| 17 | H3N2 | WT A/Wisconsin/67/2005 | Egg → MDCK (E6-P3) |
| 18 | H3N2 | WT A/Wisconsin/67/2005 | Egg → EB66 (E6-P3) |
| 19 | B | PR8 B/Malaysia/2506/2004 | Egg (E6-P0) |
| 20 | B | PR8 B/Malaysia/2506/2004 | Egg → MDCK (E6-P3) |
| 21 | B | PR8 B/Malaysia/2506/2004 | Egg → EB66 (E6-P3) |
| 22 | — | — | PBS |

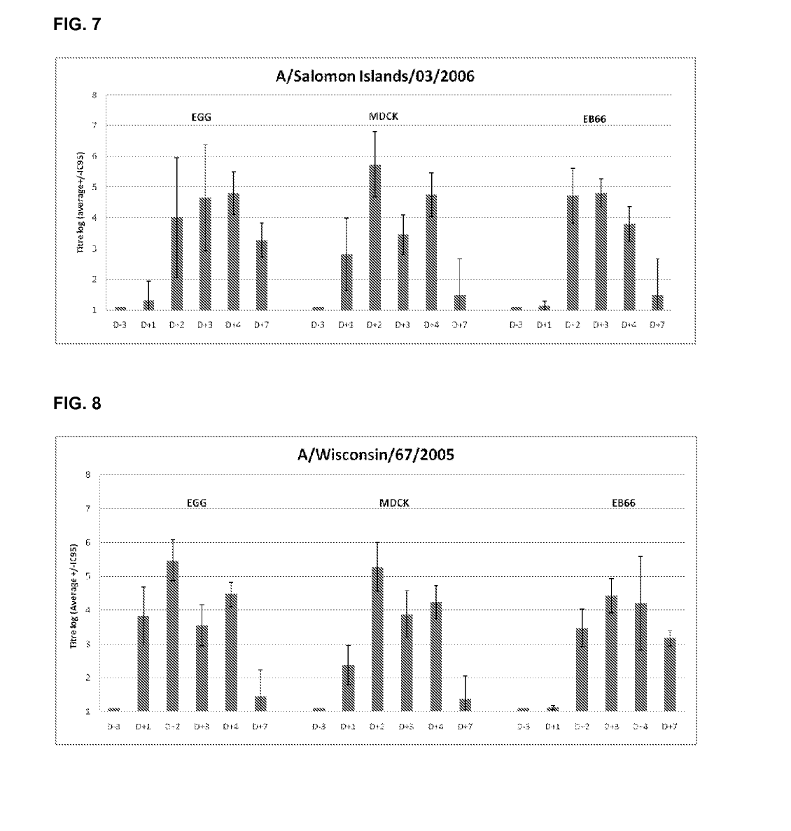

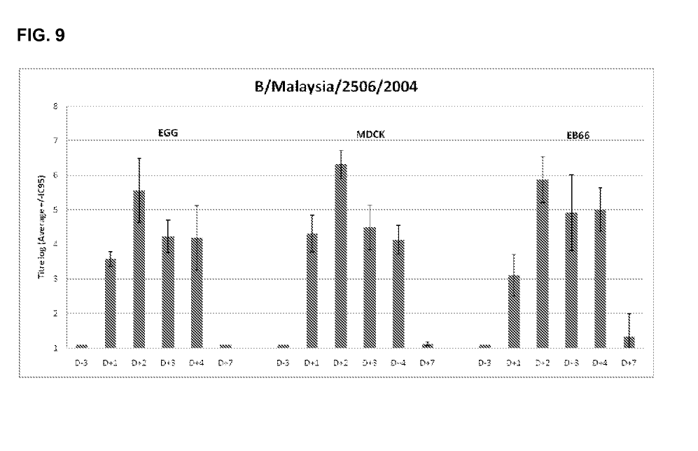

Nasal washes are collected at day 3 before and up to 7 days after challenge to measure viral replication. The viral load was assessed by viral titration on MDCK cells, the results of which are illustrated in FIGS. 7, 8 and 9.

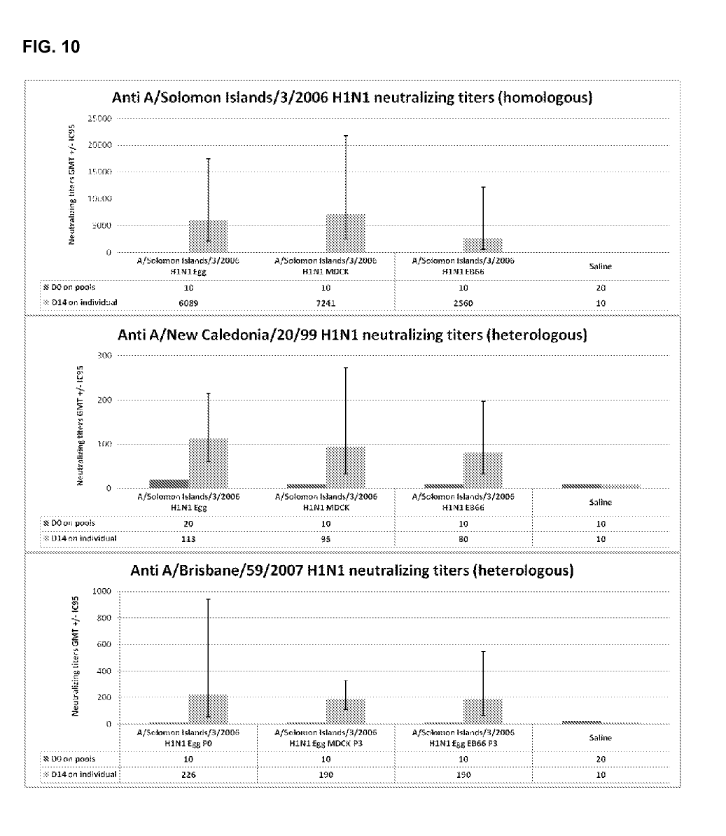

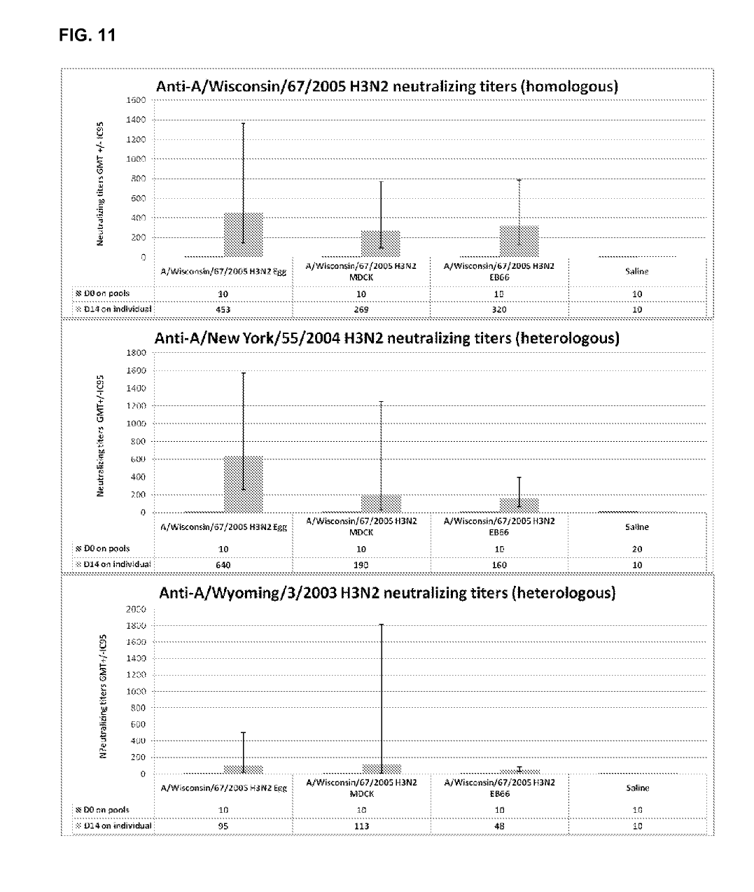

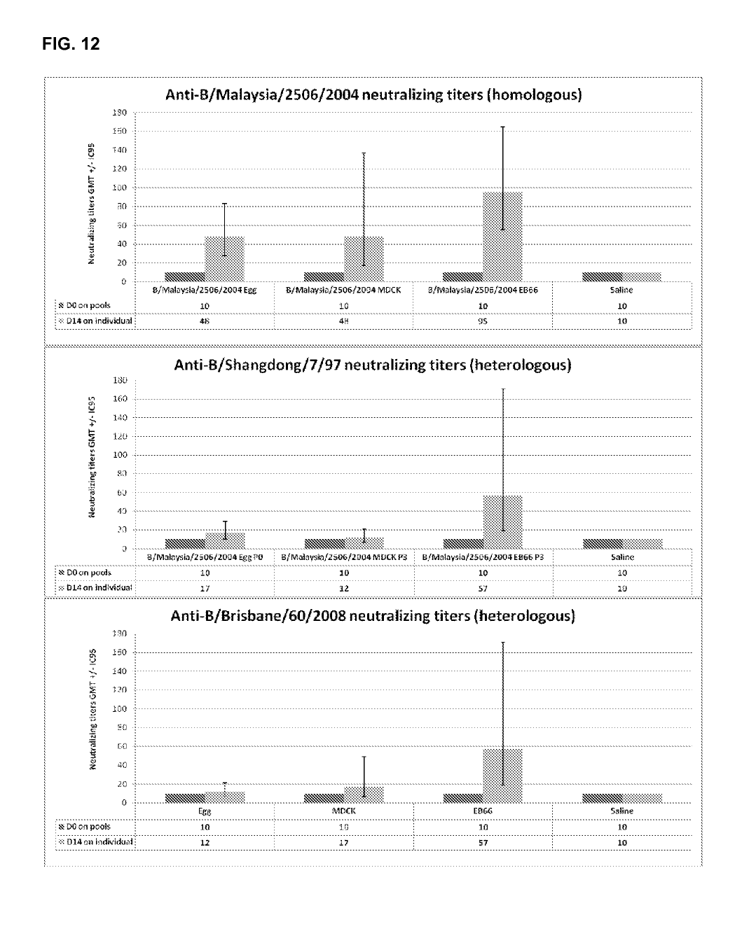

Serum samples are collected at day 0 and 14 days after challenge to measure neutralizing antibody titers against homotypic and heterosubtypic strains. Measured titers are illustrated in FIGS. 10, 11 and 12. The resulting seroprotection and seroconversion rates are listed in Tables 12 (NT titers). Seroprotection is accomplished when the titer is above 40. Seroconversion takes place when the titer post-immunization is four times higher than pre-immunization.

TABLE 12

| group | | homotypic | heterotypic | |
|---|---|---|---|---|
| | | A/Solomon | A/New | A/Brisbane/ |
| | | Islands/3/2006 | Caledonia/20/99 | 59/2007 |
| | Seroprotection | | | |
| 13 | Egg | 100% | 100% | 100% |
| 14 | MDCK | 100% | 100% | 100% |
| 15 | EB66 | 100% | 100% | 100% |
| | Seroconversion | | | |
| 13 | Egg | 100% | 100% | 100% |
| 14 | MDCK | 100% | 100% | 100% |
| 15 | EB66 | 100% | 100% | 100% |
| | | A/Wisconsin/ | A/New York/ | A/Wyoming/ |
| | | 67/2005 | 55/2004 | 3/2003 |
| | Seroprotection | | | |
| 16 | Egg | 100% | 100% | 100% |
| 17 | MDCK | 100% | 100% | 75% |
| 18 | EB66 | 100% | 100% | 100% |
| | Seroconversion | | | |
| 16 | Egg | 100% | 100% | 100% |
| 17 | MDCK | 100% | 100% | 75% |
| 18 | EB66 | 100% | 100% | 100% |
| | | B/Malaysia/ | B/Shangdong/ | B/Brisbane/ |
| | | 2506/2004 | 7/97 | 60/2008 |
| | Seroprotection | | | |
| 19 | Egg | 100% | 0% | 0% |
| 20 | MDCK | 75% | 0% | 25% |
| 21 | EB66 | 100% | 75% | 75% |
| | Seroconversion | | | |
| 19 | Egg | 100% | 0% | 0% |
| 20 | MDCK | 75% | 0% | 25% |
| 21 | EB66 | 100% | 75% | 75% |

1. A process for preparing an influenza seed virus for vaccine manufacture, comprising steps of: (i) infecting an avian cell line with an influenza virus and (ii) amplifying the virus obtained from the infected cell line of (i) to produce the influenza seed virus.

2. The process of claim 1, comprising steps of: (i) infecting a cell line with a first influenza virus; (ii) preparing a cDNA of at least one viral RNA segment of the first influenza virus produced by the infected cell line obtained in step (i), and using the cDNA in a reverse genetics procedure to prepare a new second influenza virus having at least one viral RNA segment in common with the first influenza virus of step (i); and (iii) infecting a cell line with the new second influenza virus, and (iv) amplifying the virus in order to produce a new second influenza virus for use as a seed virus.

3. The process of claim 1, wherein the influenza virus of step (i) is obtained from one of: a primary isolate in a cell line; directly from a patient; a strain circulating in the population; a strain which has a hemagglutinin that is antigenically representative of an influenza virus that is circulating in the population.

4. The process of claim 1, comprising growing the influenza virus in a mammalian cell line before infecting the avian cell line.

5. The process of claim 4, wherein the mammalian cell line is an MDCK cell line.

6. The process of claim 1, wherein the process does not comprise growing or passaging of influenza virus in eggs.

7. The process of claim 1, wherein the avian cell line is a continuous diploid avian cell line.

8. The process of claim 1, wherein the avian cell line is a duck cell line.

9. (canceled)

10. The process of claim 1, wherein the avian cell line is a stem cell line.

11. The process of claim 10, wherein the avian cell line is EB66.

12. The process of claim 1, additionally comprising the step of infecting the influenza virus into a non-human cell line after use of the avian cell line.

13. The process of claim 12, wherein the non-human cell line is an MDCK cell line.

14. The process of claim 1, wherein avian cells are the only cell culture cells used in the production of a seed vaccine.

15. The process of claim 1, wherein the avian cell line has a non-tumorigenic phenotype.

16. A process for preparing an influenza virus vaccine, comprising culturing the seed virus of claim 1 to produce influenza virus for vaccine manufacture

17. The process of claim 16, wherein the virus is treated to produce a vaccine

18. A process for making a multivalent influenza vaccine, comprising performing the process of claim 17 for an individual influenza virus strain, and mixing the product of that process with one or more other influenza virus strains, optionally made using the same process to make a multi-valent influenza vaccine.

19. A vaccine manufactured by the process of claim 17.

20. A method of treating an individual in need thereof, the method comprising vaccinating that individual using the vaccine of claim 19.

21. A process for preparing an influenza seed virus for vaccine manufacture, comprising steps of: (i) infecting a cell line with a first influenza virus; (ii) preparing a cDNA of at least one viral RNA segment of the first influenza virus produced by the infected cell line obtained in step (i), and using the cDNA in a reverse genetics procedure to prepare a new second influenza virus having at least one viral RNA segment in common with the first influenza virus of step (i); and (iii) infecting a cell line with the new second influenza virus, and (iv) amplifying the virus in order to produce a new second influenza virus for use as a seed virus, wherein the cell line is an avian cell line for one or more of steps i, iii and iv.
